(19) United States
(12) Patent Application Publication
(10) Pub. No.: US 2020/0206139 A1
(43) Pub. Date: Jul. 2, 2020

(54) COMPOSITIONS FOR THE IMPROVED DELIVERY OF DRUGS

(71) Applicant: BOARD OF REGENTS, THE UNIVERSITY OF TEXAS SYSTEM, Austin, TX (US)

(72) Inventors: Robert O. WILLIAMS, III, Austin, TX (US); Hiroyuki TAKABE, Austin, TX (US); Daniel Alan DAVIS, Austin, TX (US); Zachary WARNKEN, Austin, TX (US)

(21) Appl. No.: 16/646,452

(22) PCT Filed: Sep. 11, 2018
(86) PCT No.: PCT/US2018/050319

§ 371 Date: Mar. 11, 2020

Related U.S. Application Data
(60) Provisional application No. 62/556,991, filed on Sep. 11, 2017.

(51) Int. Cl. A61K 9/16; A61K 9/00; A61K 31/122; A61K 31/4184

(52) U.S. Cl. CPC A61K 9/1694; A61K 9/0053; A61K 31/4184; A61K 31/122; A61K 9/1652

(57) ABSTRACT

Pharmaceutical compositions for improving the solubility and dissolution of poorly soluble drugs which contain a therapeutic agent, a pharmaceutically acceptable polymer, and a spontaneously emulsifying component are described herein. These pharmaceutical compositions have been prepared by thermal processes to obtain a composition which shows improved properties including improved solubility of the therapeutic agent above the amount of therapeutic agent which should be soluble in either the spontaneously emulsifying component or the pharmaceutically acceptable polymer. Also provided herein are methods of preparing and use thereof.

The present application claims the benefit of priority to U.S. Provisional Application No. 62/556,991, filed on Sep. 11, 2017, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a drug composition containing a spontaneously emulsifying component, therapeutic agents, and pharmaceutically acceptable polymers.

### 2. Description of Related Art

The vast amount of current and in development pharmaceuticals suffer from solubility problems and thus must be formulated with one or more additional excipients to improve the solubility to obtain a clinically useful dose. As discussed by Tang et al. (2008) which is incorporated herein by reference, one particular approach to improve the solubility of these compounds is to formulate the therapeutic agents using one or more excipients which act as a self-emulsifying drug delivery system such that when the composition is added to or diluted with water or physiological fluid an emulsion is formed such that the drug remains in solution within the emulsion droplet, avoiding the dissolution step that frequently limits the absorption rate of hydrophobic drugs from the crystalline state. While these systems have been effective in improving the solubility of some agents, they are often limited by the solubility of the therapeutic agent in these compositions which can impact the therapeutic agent loading. On the other hand, amorphous solid dispersions (ASDs) allow for increased therapeutic agent loading along with increased solubility, but dissolution is typically limited by the inherent high log P values that consequently influence absorption as discussed in Friesen et al., 2008, which is incorporated herein by reference. Through the incorporation of additional excipients, limited dissolution can be overcome to create a feasible oral product, but only at the expense of a decrease in therapeutic agent loading. Thus, there is still a need to develop a formulation that contains a higher loading of the therapeutic agent, which exhibits improved wetting, dispersibility and dissolution of the therapeutic agent on dilution in physiological fluid or water.

### SUMMARY OF THE INVENTION

In some aspects, the present disclosure provides pharmaceutical compositions containing one or more therapeutic agents, a spontaneously emulsifying component containing one or more lipids, oils, or solvents and at least 1% of a surfactant, and one or more pharmaceutically acceptable polymers. These compositions may be formulated using a thermal process or fusion-based high energy mixing process that does not require an external heat input. In some aspects, the present disclosure provides pharmaceutical compositions that when added to or diluted in physiological fluid or water the therapeutic agent is present in the undissolved form.

In still another aspect, the present disclosure provides pharmaceutical composition comprising:
(A) a therapeutic agent;
(B) a pharmaceutically acceptable polymer; and
(C) a spontaneously emulsifying component, wherein the emulsifying component comprises:
   (i) a lipid, solvent, or oil; and
   (ii) at least 1% w/w relative to the weight of the composition of a surfactant or hydrophilic solvent.

In some embodiments, the pharmaceutical composition is prepared using a thermal process or a fusion-based high energy mixing process that does not require external heat input. In one embodiments, the thermal process is hot melt extrusion. Alternatively, the thermal process may be a hot melt granulation process. In some embodiments, the thermal process is carried out at a temperature below the melting point of the therapeutic agent. Additionally, the thermal process may be carried out at a temperature below the decomposition temperature of the therapeutic agent as measured by thermogravimetric analysis. In other embodiments, the composition is processed through a fusion-based high energy mixing process that does not require external heat input that results in an increase in temperature such as an increase in temperature that results from frictional or shear energy. In some embodiments, the composition has been processed by a thermokinetic mixing process.

In some embodiments, the therapeutic agent has a solubility in water of less than 5 mg/mL including therapeutic agents which are a Biopharmaceutics Classification System Class II or IV compound. Additionally, the therapeutic agent may also be known to undergo thermal degradation. In some embodiments, the therapeutic agent is known to undergo thermal degradation at a temperature greater than 80° C. The therapeutic agent may be substantially present as an amorphous form or as a molecular solution. Furthermore, the therapeutic agent may be essentially present as the amorphous form or as a molecular solution. In some embodiments, therapeutic agent is not albendazole, benomyl, benzimidazole fungicide, carbendazim, ciclobendazole, fenbendazole, flubendazole, mebendazole, nocodazole, oxfendazole and oxibendazole.

In one embodiment, the pharmaceutically acceptable polymer is a cellulosic polymer such as a neutral cellulosic polymer or an ionizable cellulosic polymer. In another embodiment, the pharmaceutically acceptable polymer is a non-cellulosic polymer such as a neutral non-cellulosic polymer or an ionizable non-cellulosic polymer. In one embodiment, the pharmaceutically acceptable polymer is a polymethacrylate or polyacrylate functionalized with a carboxylic acid group.

The methods described herein contemplate that one or more of lipids, solvent, or oils may be in the liquid phase. In some embodiments, the spontaneously emulsifying composition comprises a lipid or oil such as an ester of a fatty acid. In one embodiment, the lipid or oil is a glyceride ester of one, two, or three fatty acids such as an ester of medium chain fatty acids like Capmul®. In other embodiments, the spontaneously emulsifying composition comprises a solvent such as a hydrophobic solvent or the solvent may contain one or more aromatic groups. The solvent may be benzyl benzoate.

In one embodiment, the spontaneously emulsifying composition comprises a surfactant or hydrophilic solvent that contains one or more polyethylene glycol or polypropylene glycol repeating units. In some embodiments, the hydrophilic solvent is a PEG polymer such as a PEG polymer with a molecular weight from 100 Daltons to 2000 Daltons. Some non-limiting examples of the hydrophilic solvent are PEG 200 or PEG 400. In some embodiments, the pharmaceutical composition comprises a first surfactant. The first surfactant may be a polethoxylated castor oil such as Cremophor EL. In some embodiments, the pharmaceutical compositions further comprise a second surfactant. The second surfactant may be a compound with a hydrophobic component and a PEG or polypropylene glycol component. In one embodiment, the hydrophobic component is a fatty acid. In some embodiments, the PEG or polypropylene glycol component is a PEGylated polysorbate such as a Tween® compound.

In some embodiments, the therapeutic agent comprises from about 10% w/w to about 60% w/w of the total weight of composition, or from about 20% w/w to about 50% w/w. In one specific embodiment, the therapeutic agent comprises from about 20% w/w to about 40% w/w. In some embodiments, the therapeutic agent is present at a concentration greater than the solubility of the therapeutic agent in the pharmaceutically acceptable polymer alone. In some embodiments, the therapeutic agent is present at a concentration greater than the solubility of the therapeutic agent in the spontaneously emulsifying component alone. Additionally, the therapeutic agent may be present at a concentration greater than the solubility of the therapeutic agent in either the pharmaceutically acceptable polymer or the spontaneously emulsifying component alone. In one embodiment, the therapeutic agent is present at a concentration greater than the solubility of the therapeutic agent in either the pharmaceutically acceptable polymer or the spontaneously emulsifying component combined. In some embodiments, the therapeutic agent is formulated such that at least 10% of the therapeutic agent is present in the undissolved form when added to or diluted in physiological fluid or water. In some embodiments, at least 50% of the therapeutic agent is present in the undissolved form when added to or diluted in physiological fluid or water.

The pharmaceutically acceptable polymer may comprise from about 20% w/w to about 80% w/w of the total weight of the composition or from about 40% w/w to about 80% w/w. In some embodiments, the pharmaceutically acceptable polymer comprises from about 50% w/w to about 80% w/w. In some embodiments, the lipid, oil, or solvent comprises from about 0.25% w/w to about 10% w/w of the total weight of composition or from about 0.5% w/w to about 5% w/w. In some embodiments, the lipid, oil, or solvent comprises from about 1% w/w to about 3% w/w. The hydrophilic solvent may comprises from 2% w/w to about 10% w/w of the total weight of composition or from 2% w/w to about 6% w/w. In one embodiment, the hydrophilic solvent comprises from 3% w/w to about 5% w/w. The first surfactant may comprises from 2% w/w to about 10% w/w of the total weight of composition or from 2% w/w to about 6% w/w. In one embodiment, the first surfactant comprises from 3% w/w to about 5% w/w. In some embodiments, the composition comprises a second surfactant and the second surfactant comprises from 2% w/w to about 10% w/w of the total weight of composition or from 2% w/w to about 6% w/w. In some embodiments, the second surfactant comprises from 3% w/w to about 5% w/w.

In some embodiments, the therapeutic agent is substantially free of any crystallinity. Furthermore, the therapeutic agent may be essentially free of any crystallinity. In some embodiments, the pharmaceutical compositions exhibit a Flory-Huggins interaction parameter (χ) of greater than 0.25 as determined by differential scanning calorimetry (DSC) such as greater than 1. In other embodiments, the pharmaceutical composition exhibits a negative Flory-Huggins interaction parameter (χ) as determined by differential scanning calorimetry (DSC). In some embodiments, the pharmaceutical composition is substantially free of fatty acids. Furthermore, the pharmaceutical composition may be essentially free of any fatty acids.

In some embodiments, the pharmaceutical compositions further comprise an excipient such as a lubricant, disintegrant, binder, filler, surfactant, or any combination thereof.

In yet another aspect, the present disclosure provides method of preparing a pharmaceutical composition comprising:
(A) obtaining a composition comprising
   (1) a therapeutic agent;
   (2) a pharmaceutically acceptable polymer; and
   (3) a spontaneously emulsifying component, wherein the spontaneously emulsifying component comprises:
      (i) a lipid, solvent, or oil; and
      (ii) at least 1% w/w relative to the weight of the composition of a surfactant or a hydrophilic solvent; and
(B) heating the composition through a thermal process or a fusion-based high energy mixing process that does not require external heat input.

In some embodiments, the composition is obtained by adding the therapeutic agent, a pharmaceutically acceptable polymer, and the spontaneously emulsifying component. Alternatively, the composition may be obtained by admixing the therapeutic agent, a pharmaceutically acceptable polymer, and the spontaneously emulsifying component. In other embodiments, the composition is obtained from a third party. In some embodiments, the lipid, oil, or solvent and the surfactant or hydrophilic solvent are added together to form the spontaneously emulsifying component before addition to the therapeutic agent and the pharmaceutically acceptable polymer. In other embodiments, the spontaneously emulsifying component is mixed before addition to the therapeutic agent and the pharmaceutically acceptable polymer. The spontaneously emulsifying component may be mixed by vortexing, blending, stirring, kneading, or homogenization. In other cases, the spontaneously emulsifying component is added via a side port in the extruder during extrusion. In some embodiments, the lipid, oil, or solvent and the surfactant or hydrophilic solvent is added independently to the pharmaceutically acceptable polymer and the therapeutic agent. A further embodiments contemplates mixing the composition before heating including mixing using a homogenizer, a mixer, a vortexer, mortar and pestle, or a kneader. In other embodiments, the composition is not mixed before heating.

In one embodiments, the thermal process is hot melt extrusion. Alternatively, the thermal process may be a hot melt granulation process. In some embodiments, the thermal process is carried out at a temperature below the melting point of the therapeutic agent. Additionally, the thermal process may be carried out at a temperature below the decomposition temperature of the therapeutic agent as measured by thermogravimetric analysis. In other embodiments, the composition is processed through a fusion-based high energy mixing process that does not require external heat input that results in an increase in temperature such as an increase in temperature that results from frictional or shear energy. In some embodiments, the composition has been processed by a thermokinetic mixing process. One embodiment contemplates that the temperature of the thermal process is below the temperature required to obtain no crystallinity when formulated with the pharmaceutically acceptable polymer.

In some embodiments, the therapeutic agent comprises from about 10% w/w to about 60% w/w of the total weight of composition, or from about 20% w/w to about 50% w/w. In one specific embodiment, the therapeutic agent comprises from about 20% w/w to about 40% w/w. In some embodiments, the therapeutic agent is present at a concentration greater than the solubility of the therapeutic agent in the pharmaceutically acceptable polymer alone. In some embodiments, the therapeutic agent is present at a concentration greater than the solubility of the therapeutic agent in the spontaneously emulsifying component alone. Additionally, the therapeutic agent may be present at a concentration greater than the solubility of the therapeutic agent in either the pharmaceutically acceptable polymer or the spontaneously emulsifying component alone. In one embodiment, the therapeutic agent is present at a concentration greater than the solubility of the therapeutic agent in either the pharmaceutically acceptable polymer or the spontaneously emulsifying component combined. In some embodiments, the therapeutic agent is formulated such that at least 10% of the therapeutic agent is present in the undissolved form when added to or diluted in physiological fluid or water. In some embodiments, at least 50% of the therapeutic agent is present in the undissolved form when added to or diluted in physiological fluid or water.

The pharmaceutically acceptable polymer may comprise from about 20% w/w to about 80% w/w of the total weight of the composition or from about 40% w/w to about 80% w/w. In some embodiments, the pharmaceutically acceptable polymer comprises from about 50% w/w to about 80% w/w. In some embodiments, the lipid, oil, or solvent comprises from about 0.25% w/w to about 10% w/w of the total weight of composition or from about 0.5% w/w to about 5% w/w. In some embodiments, the lipid, oil, or solvent comprises from about 1% w/w to about 3% w/w. The hydrophilic solvent may comprises from 2% w/w to about 10% w/w of the total weight of composition or from 2% w/w to about 6% w/w. In one embodiment, the hydrophilic solvent comprises from 3% w/w to about 5% w/w. The first surfactant may comprises from 2% w/w to about 10% w/w of the total weight of composition or from 2% w/w to about 6% w/w. In one embodiment, the first surfactant comprises from 3% w/w to about 5% w/w. In some embodiments, the composition comprises a second surfactant and the second surfactant comprises from 2% w/w to about 10% w/w of the total weight of composition or from 2% w/w to about 6% w/w. In some embodiments, the second surfactant comprises from 3% w/w to about 5% w/w.

The thermal process may be hot melt extrusion which has a screw speed of the of the extruder is from about 10 rpm to about 500 rpm. In some embodiments, the screw speed is from about 50 rpm to about 250 rpm. Additionally, the thermal process may comprise heating the composition from a temperature of about 60° C. to about 220° C. or from about 100° C. to about 200° C. In some embodiments, the thermal process comprises heating the composition for a time period from about 2 minutes to about 3 hours or from about 5 minutes to about 1 hour. The methods described herein may further comprise milling the pharmaceutical composition. Additionally, the methods described herein may further comprising sieving the pharmaceutical composition such as through a screen with a pore size from about 250 μm to about 1000 μm.

In some embodiments, the methods are substantially free of a solvent. In one embodiment, the methods are essentially free of a solvent. In some embodiments, the compositions exhibit a Flory-Huggins interaction parameter (χ) of greater than 0.25 as determined by differential scanning calorimetry (DSC) such as greater than 1. In other embodiments, the composition exhibits a negative Flory-Huggins interaction parameter (χ) as determined by differential scanning calorimetry (DSC). In some embodiments, the methods is substantially free of fatty acids. Furthermore, the methods may be essentially free of any fatty acids.

In some embodiments, the therapeutic agent has a solubility in water of less than 5 mg/mL including therapeutic agents which are a Biopharmaceutics Classification System Class II or IV compound. Additionally, the therapeutic agent may also be known to undergo thermal degradation. In some embodiments, therapeutic agent is not albendazole, benomyl, benzimidazole fungicide, carbendazim, ciclobendazole, fenbendazole, flubendazole, mebendazole, nocodazole, oxfendazole and oxibendazole. In one embodiment, the pharmaceutically acceptable polymer is a cellulosic polymer such as a neutral cellulosic polymer or an ionizable cellulosic polymer. In another embodiment, the pharmaceutically acceptable polymer is a non-cellulosic polymer such as a neutral non-cellulosic polymer or an ionizable non-cellulosic polymer. In one embodiment, the pharmaceutically acceptable polymer is a polymethacrylate or polyacrylate functionalized with a carboxylic acid group.

The methods described herein contemplate that one or more of lipids, solvent, or oils may be in the liquid phase. In some embodiments, the spontaneously emulsifying composition comprises a lipid or oil such as an ester of a fatty acid. In one embodiment, the lipid or oil is a glyceride ester of one, two, or three fatty acids such as an ester of medium chain [symbol] like Capmul®. In other embodiments, the spontaneously emulsifying composition comprises a solvent such as a hydrophobic solvent or the solvent may contain one or more aromatic groups. The solvent may be benzyl benzoate.

In one embodiment, the spontaneously emulsifying composition comprises a surfactant or hydrophilic solvent that contains one or more polyethylene glycol or polypropylene glycol repeating units. In some embodiments, the hydrophilic solvent is a PEG polymer such as a PEG polymer with a molecular weight from 100 Daltons to 2000 Daltons. Some non-limiting examples of the hydrophilic solvent are PEG 200 or PEG 400. In some embodiments, the pharmaceutical composition comprises a first surfactant. The first surfactant may be a polethoxylated castor oil such as Cremophor EL. In some embodiments, the pharmaceutical compositions further comprise a second surfactant. The second surfactant may be a compound with a hydrophobic component and a PEG or polypropylene glycol component. In one embodiment, the hydrophobic component is a fatty acid. In some embodiments, the PEG or polypropylene glycol component is a PEGylated polysorbate such as a Tween® compound.

In some embodiments, the compositions further comprise an excipient such as a lubricant, disintegrant, binder, filler, surfactant, or any combination thereof.

In still some aspects, the present disclosure provides pharmaceutical compositions prepared according to the methods described herein. In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, in lipid compositions, via a catheter, via a lavage, via continuous infusion, via infusion, via inhalation, via injection, via local delivery, or via localized perfusion. The pharmaceutical compositions may be formulated for oral administration such as in a hard or soft capsule, a tablet, a syrup, a suspension, an emulsion, a solution, or a wafer.

In still another aspect, the present disclosure provides methods of treating a disease or disorder in a patient in need thereof comprising administering to the patient a therapeutically effective amount of a pharmaceutical composition described herein comprising a therapeutic agent effective to treat the disease or disorder.

In yet another aspect, the present disclosure provides methods of preventing a disease or disorder in a patient in need thereof comprising administering to the patient a therapeutically effective amount of a pharmaceutical composition described herein comprising a therapeutic agent effective to prevent the disease or disorder.

It is contemplated that any embodiment discussed in this specification can be implemented with respect to any method or composition of the invention, and vice versa. Furthermore, compositions of the invention can be used to achieve the methods of the invention.

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate, for example, that a value includes the inherent variation of error for the device, the method being employed to determine the value, or the variation that exists among the study subjects or experimental studies. In the context of this invention, “about” means approximate, and unless otherwise indicated, further means plus/minus 10%.

As used herein, the term “substantially free of” or “substantially free” in terms of a specified component, is used herein to mean that none of the specified component has been purposefully formulated into a composition and/or is present only as a contaminant or in trace amounts. The total amount of all contaminants, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows ternary phase diagram depicting regions of spontaneous emulsification of excipient combinations upon dilution in water.

FIG. 2 shows graphical representation of the dynamic light scattering result for sample A18 (see Table 2 for details about composition), showing monodisperse droplet size.

FIG. 3 shows use of Flory-Huggins theory to calculate a chi value (also called the interaction parameter) resulting in a Gibbs free energy value for PVP K30 (shown as “Polymer 1”) and spontaneous emulsifying component “A18” (shown in Table 2) at a fixed ratio of 9:1 (by weight) and ATQ at the weight fractions indicated on the X-axis. The chi parameter is used to calculate Gibbs free energy to predict if the mixture will be miscible or immiscible at the given temperature.

FIG. 4 shows a phase diagram constructed based on the Flory-Huggins modeling from FIG. 3 in order to predict drug ATQ loading and extrusion temperature.

FIG. 5 shows the chi value (as interaction parameter plotted against the 1/T, where T is the melting point at end. The slope from the curves is used to calculate the Gibbs free energy. The PVP K30 data is used in FIGS. 3 and 4. The Kollidon VA 64 data is used in FIGS. 6 and 7. The Affinisol (as Affinisol HME 15LV) data is used in FIGS. 8 and 9.

FIG. 6 shows the use of Flory-Huggins theory to calculate a chi value (also called the interaction parameter) resulting in a Gibbs free energy value for Kollidon VA 64 (shown as “Polymer 1”) and spontaneous emulsifying component “A18” (shown in Table 2) at a fixed ratio of 9:1 (by weight) and ATQ at the weight fractions indicated on the X-axis. The chi parameter is used to calculate Gibbs free energy to predict if the mixture will be miscible or immiscible at the given temperature.

FIG. 7 shows a phase diagram constructed based on the Flory-Huggins modeling from FIG. 6 in order to predict drug ATQ loading and extrusion temperature.

FIG. 8 shows the use of Flory-Huggins theory to calculate a chi value (also called the interaction parameter) resulting in a Gibbs free energy value for Affinisol HME 15LV (shown as “Polymer 1”) and spontaneous emulsifying component “A18” (shown in Table 2) at a fixed ratio of 9:1 (by weight) and ATQ at the weight fractions indicated on the X-axis. The chi parameter is used to calculate Gibbs free energy to predict if the mixture will be miscible or immiscible at the given temperature.

FIG. 9 shows a phase diagram constructed based on the Flory-Huggins modeling from FIG. 8 in order to predict drug ATQ loading and extrusion temperature.

FIG. 10 shows DSC results for the composition made of 20% ATQ and 80% PVP K30 (by weight) without the spontaneous emulsifying component. The results indicate lack of crystallinity by exhibiting no defined endotherms. The DSC method is described in example 3.

FIG. 11 shows mebendazole solubility in various pharmaceutical excipients.

FIG. 12 shows mebendazole solubility in various blends of pharmaceutical excipients. The blends of pharmaceutical excipients are described in Table 7.

FIG. 13 shows ternary phase diagram depicting regions of spontaneous emulsification of excipient combinations upon dilution in water.

FIG. 14 shows dispersibility of tablet compositions containing ATQ as compared to ATQ saturation solubility in water. Tablet compositions were prepared by Hot Melt Extrusion (20% ATQ, 10% “A18” (as described in Table 2) and 70% PVP K30 (shown as “HME 20% ATQ with i”); and 20% ATQ and 80% PVP K30 (shown as “HME 20% ATQ without i”)) and by physical mixture (20% ATQ, 10% “A18” (as described in Table 2) and 70% PVP K30. The dispersibility method is as follows: USP Apparatus 2; water is the media at 37° C., RPM 75, and 0.5 mL aliquots were taken at intervals: 5, 10, 15, 30, 60, and 120 minutes, centrifuged at 1,000 RPM for 1 minute, 0.2 mL of the supernatant was removed and then diluted with 0.8 mL of ACN, and finally assayed for ATQ by HPLC. All weights are by weight of the total composition.

FIG. 15 shows dispersibility of granules containing 20% ATQ, 10% of “A18” (from Table 2) and 70% Kollidon VA64 (shown as “20% ATQ VA64 with i”)(by weight) and 20% ATQ and 80% Kollidon VA64 (shown as “20% ATQ VA64 without”). The dispersibility method is as described in FIG. 14. All % are by weight of the total composition.

FIG. 16 shows the results of a dissolution test for mebendazole of samples analyzed at 360 minutes, which confirms degradation of mebendazole in the compositions. The compositions of Formulation 8 (Example 20), Formulation 8a (Example 21), Formulation 9 (Example 23), Formulation 10 (Example 24), Formulation 11 (Example 25) and Formulation 12 (Example 26) are described in the examples. The dissolution test method is described in the examples.

FIG. 17 shows modulated differential scanning calorimetry (mDSC) data for formulations 14, 15 and 16. The atovaquone compositions of formulations 14, 15 and 16 are described in Table 8. The mDSC conditions were as follows: 3° C./min ramp; period 60 s; and amplitude ±1° C. The results indicate that ATQ does not exhibit crystallinity by lack of a melting endotherm.

FIG. 18 shows powder X-ray diffraction results of (a) bulk atovaquone, (b) Formulation 14, (c) Formulation 15, (d) Formulation 16, (e) Formulation 17, and (f) Formulation 1. X-Ray diffraction conditions were as follows: accelerating voltage of 40 kV and 15 mA, scanned range of 0−50° at a step size of 0.05°/s and scan speed of 2°/min run on a Miniflex 600 unit.

FIG. 19 shows the results of a dispersion study. The ability of granules of atovaquone formulations 14, 15, 16 and 17 (as shown in Table 8) to wet and remain dispersed in dissolution media following centrifugation (1000 rpm for 1 min) is shown. The dispersion test was conducted using USP Apparatus 2 (paddle) and the following test conditions: deionized water (900 mL); Temperature—37 C; paddle speed—100 rpm; samples times as indicated on the X-axis. The amount of atovaquone from the granules remaining dispersed following centrifugation (1000 rpm for 1 min) was determined by HPLC.

FIG. 20 shows the results of a biphasic dissolution test. The ability of granules of Formulations 14, 15, 16 and 17 (as shown in Table 8) to release atovaquone into the aqueous dissolution media (either as a small particle or dissolved) and dissolve into the octanol phase. Aliquots of 300 mg of each formulation was added to 700 mL of water phase. Then, 200 mL of octanol was added to create the biphasic dissolution media. The test was performed at a paddle speed of 200 RPM at 37° C. The granules of Formulations 14, 15 and 16 immediately wet and disperse. Atovaquone concentrations were directly sampled from the octanol phase at time points indicated on the X-axis and filtered using a 0.22 micron PTFE filter, diluted with ACN, and assayed by HPLC.

FIG. 21 shows a biphasic dissolution test. Formulation 18 was prepared as follows. Atovaquone, PVP K30, benzyl benzoate, Tween 20 and PEG 400 (20:70:3:4:3 w/w) were blended together and then thermally processed using a Mini Haake compounder. The conditions were: 180° C., 150 rpm screw speed; batch size 10 g. The extrudate was cooled to room temperature and then mechanically milled using a grinder to form granules. The granules were passed through a 600 micron sieve. For comparison, dissolution results for Formulation 17 (as described in Table 8) are plotted as well.

FIG. 22 shows atovaquone concentrations in mice sera over time after dosing with either Formulation 14 or Formulation 17. Error bars represent the standard deviation from n=3 mice.

### DESCRIPTION OF ILLUSTRATIVE EMBODIMENTS

In some aspects of the present disclosure, the pharmaceutical compositions prepared through thermal processing or fusion-based high energy processing that does not require external heat input and containing a therapeutic agent, a pharmaceutically acceptable polymer, and a spontaneously emulsifying component which show improved wetting, dispersibility, dissolution or other pharmaceutical properties are provided. These compositions may show improved solubility parameters (e.g., expanded range of polymer and drug miscibility, lower temperature for improved miscibility) and exhibit higher therapeutic agent loading than the that of the therapeutic agent in any of the other individual components. Additionally, the pharmaceutical compositions described herein may contain at least 1% of a surfactant, such as a non-ionic surfactant. Furthermore, the choice of polymer may be determined by using such parameters as Flory-Huggins theory to predict and guide the specific pairing of the therapeutic agent and the pharmaceutically acceptable polymer. Also provided herein are methods of preparing and using these compositions. Details of these compositions are provided in more detail below.

### I. Pharmaceutical Compositions

In some aspects, the present disclosure provides pharmaceutical compositions containing a therapeutic agent, a pharmaceutically acceptable polymer, and a spontaneously emulsifying component which contains a lipid, oil, or solvent and at least 1% of a surfactant and have been processed through a thermal process or fusion-based process. In some embodiments, the thermal process may be a hot melt extrusion or a hot melt granulation process. In other embodiments, the fusion-based high energy process is a process which results in an increase in temperature without requiring an external heat input including thermokinetic mixing process such as those described in U.S. Pat. Nos. 8,486,423; 9,339,441; Prasad et al., 2016; LaFountaine et al., 2016; and DiNunzio et al., 2010d. Additionally, these pharmaceutical compositions may show improved solubility or dissolution profiles which result in one or more improved therapeutic parameters.

Additionally, the present pharmaceutical composition may have the added benefit of not requiring the mixing or milling of the components of the composition before being subjected to the thermal or fusion-based high energy process. Such advantages simplifies the formulation process and reduces possible likelihood of drug decomposition or degradation. Additionally, the loading of the therapeutic agent may be higher than the solubility of the therapeutic agent in either of the pharmaceutically acceptable polymer or the spontaneously emulsifying component, or both. Additionally, in some embodiments, the present disclosure provides pharmaceutical compositions that when added to or diluted in physiological fluid or water the therapeutic agent is present in the undissolved form.

A. Therapeutic Agent

The “therapeutic agent” used in the present methods and compositions refers to any substance, compound, drug, medicament, or other primary active ingredient that provides a therapeutic or pharmacological effect when administered to a human or animal. Some non-limiting examples of lipophilic therapeutic agents are BCS classes II and IV compounds or other agents that similarly exhibit poor solubility. The BCS definition describes a compound in which the effective dosing is not soluble in 250 mL of water at a pH from 1-7.5. The USP categories “very slightly soluble” and “insoluble” describe a material that requires 1,000 or more parts of the aqueous liquid to dissolve 1 part solute. As used herein, when a compound is described as poorly soluble, it refers to a compound which has solubility in water of less than 1 mg/mL. In other embodiments, the therapeutic agent is an active agent which has a high melting point. Some non-limiting examples of high melting point therapeutic agents are griseofulvin and theophylline.

When a therapeutic agent is present in the composition, the therapeutic agent is present in the composition at a level between about 10% to about 60% w/w, between about 20% to about 50% w/w, between about 20% to about 40% w/w, between about 30% to about 60% w/w, or between about 40% to about 60% w/w of the total composition. In some embodiments, the amount of the therapeutic agent is from about 10%, 15%, 16%, 17%, 18%, 19%, 20%, 21%, 22%, 23%, 24%, 25%, 26%, 27%, 28%, 29%, 30%, 32%, 34%, 35%, 36%, 38%, 40%, 45%, 50%, 55%, to about 60% w/w or any range derivable therein. In some embodiments, the amount of the therapeutic agent in the composition is higher than the solubility of the therapeutic agent in any of the components of the spontaneously emulsifying component or any part of this component. In some embodiments, the amount of the therapeutic agent in the composition is higher than the solubility of the therapeutic agent in any of the pharmaceutically acceptable polymer. Alternatively, the amount of the therapeutic agent in the composition is higher than the solubility of the therapeutic agent in the spontaneously emulsifying component and the pharmaceutically acceptable polymer combined. As contemplated herein, a portion of the therapeutic agent may be undissolved in the pharmaceutical composition once the pharmaceutical composition has been added to or diluted in physiological fluid or water. In some embodiments, the therapeutic agent is formulated such that at least 10% of the therapeutic agent is present in the undissolved form when added to or diluted in physiological fluid or water. Furthermore, the pharmaceutical composition may comprise at least about 5%, 10%, 15%, 20%, 25%, 30%, 35%, 40%, 45%, 50%, 55%, 60%, 65%, 70%, 75%, 80%, 85%, 90%, 95%, or about 99% of the therapeutic agent is present in the undissolved form when added to or diluted in physiological fluid or water.

Suitable lipophilic therapeutic agents may be any poorly water-soluble, biologically active agents or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the therapeutic agents may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the therapeutic agents may be busulfan, taxane or other anticancer agents; or alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole and c) thiazoles such as abafungin. Other drugs that may be used with this approach include, but are not limited to, hyperthyroid drugs such as carimazole, anticancer agents like cytotoxic agents such as epipodophyllotoxin derivatives, taxanes, bleomycin, anthracyclines, as well as platinum compounds and camptothecin analogs. The following therapeutic agents may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The agents may also include a psychiatric agent such as an antipsychotic, anti-depressive agent, or analgesic and/or tranquilizing agents such as benzodiazepines. The agents may also include a consciousness level-altering agent or an anesthetic agent, such as propofol. The present compositions and the methods of making them may be used to prepare a pharmaceutical compositions with the appropriate pharmacokinetic properties for use as therapeutics.

Without wishing to be bound by any theory, it is believe that some drugs are not amenable to this technology due to severe chemical degradation, which makes the subsequent compositions made as described herein containing these drugs not pharmaceutically acceptable. These drugs include, for example, Albendazole, Benomyl, Benzimidazole fungicide, Carbendazim, Ciclobendazole, Fenbendazole, Flubendazole, Mebendazole, Nocodazole, Oxfendazole and Oxibendazole.

In some aspects, the method may be most used with materials which undergo degradation at an elevated temperature or pressure. The therapeutic agents that may be used include those which decompose at a temperature above about 50° C. In some embodiments, the therapeutic agent decomposes above a temperature of 80° C. In some embodiments, the therapeutic agent decomposes above a temperature of 100° C. In some embodiments, the therapeutic agent decomposes above a temperature of 150° C. The therapeutic agent that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

B. Pharmaceutically Acceptable Polymers

In some aspects, the present disclosure provides compositions which may further comprise a pharmaceutically acceptable polymer. In some embodiments, the polymer has been approved for use in a pharmaceutical formulation and is known to undergo softening or increased pliability when raised above a specific temperature without substantially degrading. Additionally, the pharmaceutically acceptable polymer may also be known to enhance the dissolution of one or more of the therapeutic agents in the composition or pharmaceutical composition.

When a pharmaceutically acceptable polymer is present in the composition, the pharmaceutically acceptable polymer is present in the composition at a level between 40% to 80% w/w, between 50% to 80% w/w, between 60% to 80% w/w, between 40% to 50% w/w, between 50% to 60% w/w of the total pharmaceutical composition. In some embodiments, the amount of the pharmaceutically acceptable polymer is from about 40%, 42%, 44%, 45%, 46%, 48%, 50%, 52%, 54%, 55%, 56%, 58%, 60%, 62%, 64%, 65%, 66%, 68%, 70%, 72%, 74%, 75%, 76%, 78%, to about 80% w/w or any range derivable therein. In some embodiments, the amount of the therapeutic agent in the composition is higher than the solubility of the therapeutic agent in the pharmaceutically acceptable polymer.

In some aspects, Flory-Huggins theory can be used as a preformulation test to guide or predict appropriate therapeutic agent and pharmaceutically acceptable polymer combination and their miscibility and physical stability (Lu et al., 2015; Qian et al., 2010). Flory-Huggins theory may be applied to determine miscibility information for amorphous drug-polymer systems by evaluating the drug-polymer interaction parameter, χ, to calculate the free energy of mixing (ΔGₘᵢₓ) for the system. The χ value stems from the non-ideal entropy of mixing of the pharmaceutically acceptable polymer molecule with the therapeutic agent and takes into account the contribution due to the enthalpy of mixing (Bansal et al., 2016). More negative χ values predict miscibility whereas more positive χ values predict immiscibility of the therapeutic agent-polymer system (Bansal et al., 2016; Marsac et al., 2006). According to Flory-Huggins theory,

$$Δ {G}_{mix} = RT ( {Φ}_{drug} ln {Φ}_{drug} + \frac{{Φ}_{polymer}}{m} ln {Φ}_{polymer} + {χΦ}_{drug} {Φ}_{polymer} ) \qquad ( Equation 1 )$$

where Φ is the volume fraction, χ is the Flory-Huggins interaction parameter, R is the molar gas constant, and T is the temperature. m is the ratio of the volume of a pharmaceutically acceptable polymer to the therapeutic agent molecular volume and,

$$m = \frac{\frac{{MW}_{polymer}}{{ρ}_{polymer}}}{\frac{{MW}_{drug}}{{ρ}_{drug}}} \qquad ( Equation 2 )$$

where the MW_{polymer} and MW_{drug} are the molecular weight of the pharmaceutically acceptable polymer and therapeutic agent, respectively, and the ρ_{polymer} and ρ_{drug} are the density of pharmaceutically acceptable polymer and therapeutic agent, respectively. The primary method for determining the χ value is by analyzing the melting point depression of the solid dispersion system, which is often, analyzed using differential scanning calorimetry (DSC). DSC is utilized to determine the melting point onset (Zhao et al., 2011), melting temperature (Lin and Huang, 2010; Marsac et al., 2008), or melt endpoint (Tian et al., 2013). Following analysis of melting point depressions, the χ value can be calculated using the following rearranged equation (Marsac et al., 2006).

$$( \frac{1}{{T}_{M}^{mix}} - \frac{1}{{T}_{M}^{pure}} ) ( \frac{Δ {H}_{fus}}{- R} ) - ln {Φ}_{drug} - ( 1 - \frac{1}{m} ) {Φ}_{polymer} = {χΦ}_{polymer}^{2} \qquad ( Equation 3 )$$

where T_{M} values are the melting points of the mixture of pure therapeutic agent, R is the ideal gas constant, ΔH_{fus} is the heat of fusion for the pure therapeutic agent, m is a constant for the relative size of the pharmaceutically acceptable polymer to the therapeutic agent, and the Φ values are volume fraction of therapeutic agent or pharmaceutically acceptable polymer. If the plot of the left side of the rearranged equation vs. the Φ² value for the pharmaceutically acceptable polymer demonstrates linearity, the slope of the best-fit line is considered to be equivalent to χ. By understanding χ as a function of temperature, metastable and unstable regions for the combination can be predicted by generating a spinodal (boundary between unstable and metastable regions) and binodal (boundary between metastable and stable regions) curves (Huang et al., 2016). If the solid dispersion system's components are stable, these systems tend to remain in a single-phase, while metastable and unstable systems tend to phase separate into drug-rich and polymer-rich domains upon storage. Without wishing to be bound by any theory it is believed that the tendency to recrystallize occurs because the high-energy amorphous state is generally unstable (Marsac et al., 2010; Purohit and Taylor, 2015). Therefore, in some embodiments, Flory-Huggins theory as a preformulation test contemplates that the combination of the pharmaceutically acceptable polymer and the therapeutic agent exhibits a stable combination. In other aspects, the present combinations of the pharmaceutically acceptable polymer and the therapeutic agent exhibits a positive χ value.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups which are charged at a physiologically relevant pH. Some non-limiting examples of neutral non-cellulosic polymers include polyvinyl pyrrolidone, polyvinyl alcohol, copovidone, poloxamer, polyethylene oxide, polypropylene oxide, polyvinylpyrrolidone-co-vinylacetate, polyethylene, polycaprolactone, and polyethylene-co-polypropylene. Some examples of ionizable non-celluolosic polymers include polymethacrylate or polyacrylate such as Eudragit®. Some non-limiting examples of ionizable cellulosic polymers include hydroxyalkylalkyl cellulose ester such as cellulose acetate phthalate and hydroxypropyl methyl cellulose acetate succinate, carboxyalkyl cellulose such as carboxymethyl cellulose and alkali metal salts thereof, such as sodium salts, and carboxyalkylalkyl cellulose including carboxymethylethyl cellulose, carboxyalkyl cellulose ester such as carboxymethyl cellulose butyrate, carboxymethyl cellulose propionate, carboxymethyl cellulose acetate butyrate, and carboxymethyl cellulose acetate propionate. Finally, some non-limiting examples of neutral cellulosic polymers include alkylcelluloses such as methylcellulose, hydroxyalkylcelluloses such as hydroxymethylcellulose, hydroxypropyl cellulose, hydroxyethylcellulose, and hydroxybutylcellulose, hydroxy alkyl alkylcelluloses such as hydroxyethyl methylcellulose and hydroxypropyl methyl cellulose, starches, pectins, chitosan or chitin and copolymers and mixtures thereof, and polysaccharides such as tragacanth, gum arabic, guar gum, and xanthan gum.

Some specific pharmaceutically acceptable polymers which may be used include, for example, Eudragit™ RS PO, Eudragit™ 5100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55 ™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

C. Spontaneously Emulsifying Component

In some aspects, the present disclosure comprises a spontaneously emulsifying component comprising a mixture of one or more lipids, oils, or solvents and one or more surfactants. In one embodiment, the spontaneously emulsifying component contains at least one surfactant which comprises at least 1% w/w of the total weight of the pharmaceutical composition. In some embodiments, the amount of the therapeutic agent in the composition is higher than the solubility of the therapeutic agent in the mixture of the components of the spontaneously emulsifying component. The amount of the therapeutic agent in the composition may be higher than about two times, three times, four times, or five times the therapeutic agent's solubility in the spontaneously emulsifying component.

In some aspects, the amount of spontaneously emulsifying component is from about 1.25% to about 40% w/w. The amount of spontaneously emulsifying component comprises from about 1%, 1.25%, 1.5%, 1.75%, 2%, 3%, 4%, 5%, 6%, 7%, 8%, 9%, 10%, 12%, 14%, 15%, 16%, 18%, 20%, 22%, 24%, 25%, 26%, 28%, 30%, 32%, 34%, 35%, 36%, 38%, to about 40% w/w, or any range derivable therein, of the total pharmaceutical composition. In one embodiment, the amount of spontaneously emulsifying component is at 5 to 15% w/w of the total weight of the pharmaceutical composition.

A. Lipid, Oil, and Solvents

In some aspects, the present disclosure provides pharmaceutical compositions containing one or more lipid, oil, or solvent. As used herein, a lipid or oil is a hydrophobic molecule which exhibits fat-like solubility and is largely insoluble in water. The lipid or oil may be a fatty acid, a triglyceride, an ester of a fatty acid, or mixtures thereof. The term lipid includes fatty acids which are a group of aliphatic saturated or unsaturated carboxylic acids. The chains are usually unbranched and have 6 to 30, preferably 8 to 22, and in particular 8 to 18, carbon atoms. Some non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotic acid and melissic acid. Additionally, the term includes unsaturated, fatty acids may be unsaturated one or more times, in particular unsaturated once, twice, three times, four times, five times or six times. Some non limiting examples of singly unsaturated fatty acids include palmitoleic acid, oleic acid and erucic acid, of doubly unsaturated fatty acids include sorbic acid and linoleic acid, of triply unsaturated fatty acids include linolenic acid and eleostearic acid, of quadruply unsaturated fatty acids include arachidonic acid, of quintuply unsaturated fatty acids include clupanodonic acid, and of sextuply unsaturated fatty acids include docosahexaenoic acid.

Alternatively, the terms lipid and oil may include glycerides which are esters of glycerol. Depending on the number of ester groups, the glyceride may be referred to as a mono-, di- and triglycerides. The acid, residue in a monoglyceride may be at position 1 or 2 and the acid residues of di- and triglycerides may be identical or different and be distributed in every conceivable way over the three possible positions of glycerol. The acid residues are preferably the fatty acids described above. Examples of monoglycerides include glycerol monobehenate, glycerol monocaprate, glycerol monococoate, glycerol monoerucate, glycerol monoisostearate, glycerol monolanolate, glycerol monolaurate, glycerol monolinoleate, glycerol monomyristate, glycerol monooleate, glycerol monopalmitate, glycerol monoricinoleate, glycerol monostearate, of the diglycerides include glycerol dicaprylate, glycerol dilaurate, glycerol dimyristate, glycerol dioleate, glycerol dipalmitate and glycerol distearate, of the triglycerides include glycerol tricaprylate, glycerol trilaurate, glycerol trimyristate, glycerol trioctanoate, glycerol trioleate, glycerol triricinoleate and glycerol tristearate. Many common pharmaceutical oils and lipids comprises one or more glycerides and these pharmaceutical oils and lipids include Capmul, CapTex, and Labrafac. Additionally, other oils or lipids may include esters of fatty acids such as methyl palmitate, ethyl linoleate, or isopropyl palmitate.

In other aspects, the present disclosure provides pharmaceutical compositions which comprises one or more solvents. The solvents which may be used in the present compositions include hydrophobic solvents which are liquid at room temperature. In particular, the solvent may contain one or more aromatic rings and/or have a high boiling point. Some non-limiting solvents for the pharmaceutical compositions described herein include benzyl benzoate and benzyl alcohol.

In some aspects, the amount of each lipid, oil, or solvent is from about 1% to about 20% w/w, from about 2% to about 10% w/w, from about 2% to about 8% w/w, or from about 2% to about 4% w/w. The amount of each lipid, oil, or solvent comprises from about [symbol] , 1.25%, 1.5%, 1.75%, 2%, 3%, 4%, 5%, 6%, 7%, 8%, 9%, 10%, 12%, 14%, 15%, 16%, 18%, to about 20% w/w, or any range derivable therein, of the total pharmaceutical composition. In one embodiment, the amount of each lipid, oil, or solvent is at 2% to 5% w/w of the total weight of the pharmaceutical composition.

B. Surfactants or Hydrophilic Solvents

In some aspects, the present disclosure provides pharmaceutical compositions comprising spontaneously emulsifying component which have one or more surfactants. In some embodiments, the compositions have either one or two surfactants. As used herein, the term surfactant refers to a compound which exhibits amphiphilic character and reduces the surface tension of a solvent, particularly water. Surfactants can generally be classified into four categories: cationic, anionic, zwitterionic, or non-ionic. While it is contemplated that any of these surfactants may be used in the present compositions, non-ionic surfactant show particular promise. Cationic surfactants include, but are not limited to, amines with long alkyl chains and are protonated at a physiologically relevant pH or permanently charged quaternary ammonium salts such as cetrimonium bromide, cetylpyridinium chloride, benzalkonium chloride, benzethonium chloride, dimethyldioctadecylammonium chloride, or dioctadecyldimethylammonium bromide. Some non-limiting examples of anionic surfactants include sulfate, sulfonate, or phosphate esters such as docusate, perfluorooctanesulfonate, perfluorobutanesulfonate, alkyl-aryl ether phosphates, or alkyl ether phosphate or carboxylate esters including alipahtic carboxylates such as fatty acids and derivatives thereof. Other examples of zwitterionic surfactants including phospholipids such as phosphotidylserine, phosphotidylcholine, phosphotidylethanolamine, or sphingomyelins, sultaines such as CHAPS and cocamidopropyl hydroxysultaine, or betaine such as cocamidopropyl betaine. Finally, some non-limiting examples of nonionic surfactants include PEG alkyl ethers, polypropylene glycol ethers, glucoside alkyl ethers, PEG alkylaryl ethers such as Triton® and nonoxynol, simple alkyl esters of glycerol such as glycerol laurate, polysorbates such as Tween, Sorbitan alkyl esters such as Span, or poloxamer and other block copolymers of polyethylene glycol and polypropylene glycol. In some embodiments, the surfactants used in the present pharmaceutical compositions contain one or more polyethylene glycol or polypropylene glycol polymer such as Tween, Capryol, Labrafil, or Labrasol.

In some aspects, the amount of the first or second surfactant is from about 1% to about 20% w/w, from about 2% to about 10% w/w, from about 2% to about 8% w/w, or from about 2% to about 4% w/w. The amount of the first or second surfactant comprises from about 1%, 1.25%, 1.5%, 1.75%, 2%, 3%, 4%, 5%, 6%, 7%, 8%, 9%, 10%, 12%, 14%, 15%, 16%, 18%, to about 20% w/w, or any range derivable therein, of the total pharmaceutical composition. In one embodiment, the amount of the first or second surfactant is at 2% to 5% w/w of the total weight of the pharmaceutical composition.

In other aspects, the present disclosure comprises one or more hydrophilic solvents. Such hydrophilic solvents are ready water miscible compounds. These hydrophilic solvents include alcohols such as ethanol or isopropanol, polyethylene glycol (e.g. PEG), propylene glycol, or diethylene glycol monoethyl ether (Transcutol). In some embodiments, the hydrophilic solvent is a PEG polymer with a molecular weight from about 100 to about 4000 daltons, from about 100 to about 1000 daltons, from about 100 to about 500 daltons, or from about 100, 200, 300, 400, 500, 600, 700, 800, 900, 1000, 1250, 1500, 1750, 2000, 2500, 3000, 3500, or about 4000 daltons.

In some aspects, the amount of the hydrophilic solvent is from about 1% to about 20% w/w, from about 2% to about 10% w/w, from about 2% to about 8% w/w, or from about 2% to about 4% w/w. The amount of the hydrophilic solvent comprises from about 1%, 1.25%, 1.5%, 1.75%, 2%, 3%, 4%, 5%, 6%, 7%, 8%, 9%, 10%, 12%, 14%, 15%, 16%, 18%, to about 20% w/w, or any range derivable therein, of the total pharmaceutical composition. In one embodiment, the amount of the hydrophilic solvent is at 2% to 5% w/w of the total weight of the pharmaceutical composition.

### II. Manufacturing Methods

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher, such as a minilab compounder, or Leistritz, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the extruder may comprise heating the composition to a temperature from about 60° C. to about 250° C. In some embodiments, the temperature is from about 100° C. to about 200° C. The temperature that may be used is from about 60° C., 65° C., 70° C., 75° C., 80° C., 90° C., 92° C., 94° C., 96° C., 98° C., 100° C., 102° C., 104° C., 106° C., 108° C., 110° C., 112° C., 114° C., 116° C., 118° C., 120° C., 125° C., 130° C., 135° C., 140° C., 145° C., 150° C., 155° C., 160° C., 165° C., 170° C., 175° C., 180° C., 190° C., 200° C., 225° C., to about 250° C. or any range derivable therein.

The extrudate produced following the extrusion process will generally comprise the therapeutic agent, the spontaneously emulsifying component, and the pharmaceutically acceptable polymer. The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final table. Additionally, it may be milled and combined with one or more additional excipients to form a capsule or pressed into a table. The resultant pharmaceutical composition may also be dissolved in a solvent to obtain a syrup, a suspension, an emulsion, or a solution.

### III. Examples

To facilitate a better understanding of the present invention, the following examples of specific embodiments are given. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. In no way should the following examples be read to limit or define the entire scope of the invention.

### Example 1—Atovaquone Solubility in Pharmaceutical Excipients

Excess atovaquone (100 mg/mL) (Molekula, Irvine, Calif.) was added to each of the following excipients: Benzyl Benzoate, Tween 20, Tween 80, PEG 200, PEG 400, Labrafac, Labrafil, Capryol, Capmul, Captex, Propylene Glycol and Isopropyl Palmitate. The mixtures were shaken at 100 RPM at 37° C. for 48 hours. Each individual sample was centrifuged at 3000 RPM for 10 minutes. The supernatant was then filtered through a 0.45 μm PTFE filter. Quantification of atovaquone content in the filtrate was performed using a Dionex HPLC (Thermo Fisher Scientific Inc., Waltham, Mass.) with an Inertsil® ODS-2 column (5 μm, 4.6×250 mm) with a mobile phase 0.1% TFA ACN: 0.1% TFA H₂O (80:20) at flow rate of 2 mL/min and a run time of 10 minutes. The results of the solubility study are shown in Table 1.

TABLE 1
Results of solubility study for atovaquone.

| Excipients | mg/mL | S.D. |
|---|---|---|
| Benzyl Benzoate | 14.31 | 0.41 |
| Tween 20 | 9.69 | 0.74 |
| Tween 80 | 10.22 | 1.22 |
| PEG 200 | 4.78 | 0.21 |
| PEG 400 | 7.22 | 0.71 |
| Labrafac | 4.08 | 0.46 |
| Labrafil | 3.98 | 0.23 |
| Labrasol | 10.23 | 0.5 |
| Capryol | 5.54 | 0.43 |
| Capmul | 3.47 | 0.31 |
| Captex | 4.59 | 0.58 |
| Proplylene Gycol | 0.38 | 0.03 |
| Isopropyl Palmitate | 2.46 | 0.08 |

### Example 2—Preformulation Studies for Characterizing the Spontaneous Emulsifying Component

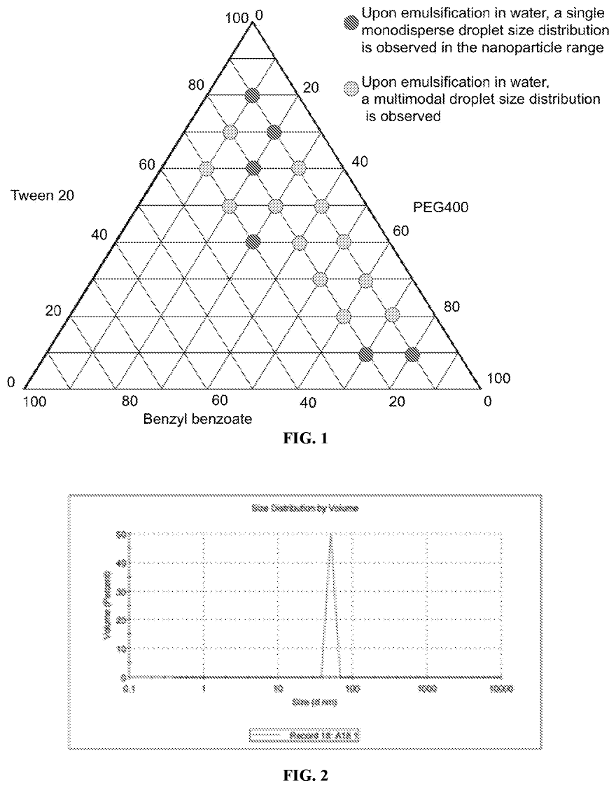

36 unique combinations were made from mixing PEG 400, Tween 20 and Benzyl Benzoate in different ratios. 10 mcL of each formulation was added to 10 mL of water and vortexed. Emulsification was determined by visual inspection and distribution confirmed by Zetasizer Nano ZS (Malvern Instruments, Worcestershire, UK). Results are depicted as a ternary phase diagram in FIG. 1. Excipient combinations employed are shown in Table 2. Particle size distribution data for various excipient combinations is shown in Table 3. Particle (droplet) size results by dynamic light scattering results for sample A18 are shown in FIG. 2. Solubility of Atovaquone in the final excipient combinations was confirmed by the method described in Example 2.

TABLE 2
Excipient combinations.

| Excipient | A1 | A2 | A3 | A4 | A5 | A6 | A7 | A8 | A9 | A10 | A11 | A12 | A13 | A14 | A15 | A16 | A17 | A18 |
|---|---|---|---|---|---|---|---|---|---|---|---|---|---|---|---|---|---|---|
| PEG400 | 80 | 70 | 70 | 60 | 60 | 60 | 50 | 50 | 50 | 50 | 40 | 40 | 40 | 40 | 40 | 30 | 30 | 30 |
| Tween20 | 10 | 20 | 10 | 30 | 20 | 10 | 40 | 30 | 20 | 10 | 50 | 40 | 30 | 20 | 10 | 60 | 50 | 40 |
| Benzyl Benzoate | 10 | 10 | 20 | 10 | 20 | 30 | 10 | 20 | 30 | 40 | 10 | 20 | 30 | 40 | 50 | 10 | 20 | 30 |
| Excipient | A19 | A20 | A21 | A22 | A23 | A24 | A25 | A26 | A27 | A28 | A29 | A30 | A31 | A32 | A33 | A34 | A35 | A36 |
| PEG400 | 30 | 30 | 30 | 20 | 20 | 20 | 20 | 20 | 20 | 20 | 10 | 10 | 10 | 10 | 10 | 10 | 10 | 10 |
| Tween20 | 30 | 20 | 10 | 70 | 60 | 60 | 40 | 30 | 20 | 10 | 80 | 70 | 60 | 50 | 40 | 30 | 20 | 10 |
| Benzyl Benzoate | 48 | 50 | 60 | 10 | 20 | 30 | 48 | 50 | 60 | 70 | 10 | 20 | 30 | 40 | 50 | 60 | 70 | 80 |

TABLE 3
Particle size distribution by dynamic light scattering for each combination of excipients.

| sample | | | | | | |
|---|---|---|---|---|---|---|
| name | size (d.nm) | % volume | size (d.nm) | % volume | size (d.nm) | % volume |
| A1 | 124 | 100 | | | | |
| A2 | 68.12 | 23.6 | 228.6 | 73.3 | 5469 | 3.2 |
| A3 | 332.6 | 100 | | | | |
| A4 | 89.01 | 49.5 | 251.8 | 50.5 | | |
| A5 | 229.4 | 69.2 | 66.69 | 40.8 | | |
| A6 | 497.8 | 100 | | | | |
| A7 | 203 | 74.6 | 5064 | 5 | 40.92 | 20.5 |
| A8 | 217.3 | 65.1 | 51.22 | 34.9 | | |
| A9 | 44.06 | 100 | | | | |
| A10 | 578.6 | 100 | | | | |
| A11 | 99.11 | 36.3 | 307.4 | 60.4 | 5202 | 3.3 |
| A12 | 91.36 | 37.8 | 288.1 | 62.2 | | |
| A13 | 175.6 | 40.6 | 659.4 | 36.2 | 44.02 | 20.1 |
| A14 | 3.893 | 100 | | | | |
| A15 | | | | | | |
| A16 | 178.6 | 72.1 | 38.67 | 27.9 | | |
| A17 | 107 | 25.6 | 349.5 | 87.5 | 5385 | 6.9 |
| A18 | 51.02 | 100 | | | | |
| A19 | 116.2 | 32.4 | 207.2 | 21.9 | 466.6 | 46.5 |
| A20 | 32.65 | 100 | | | | |
| A21 | | | | | | |
| A22 | 92.22 | 100 | | | | |
| A23 | 242.2 | 100 | | | | |
| A24 | 287.6 | 77.6 | 65.71 | 22.2 | | |
| A25 | 43.15 | 100 | | | | |
| A26 | | | | | | |
| A27 | 158.4 | 100 | | | | |
| A28 | | | | | | |
| A29 | 113.8 | 100 | | | | |
| A30 | 61.04 | 22.5 | 236.3 | 77.5 | | |
| A31 | 226.6 | 91.6 | 5401 | 6.4 | | |
| A32 | 57.8 | 51 | 241.1 | 49 | | |
| A33 | 54.57 | 12.6 | 198.5 | | | |
| A34 | 163.9 | 100 | | | | |
| A35 | | | | | | |
| A36 | 1.745 | 100 | | | | |

### Example 3—Flory-Huggins Modeling for Atovaquone and Povidone K30 Miscibility

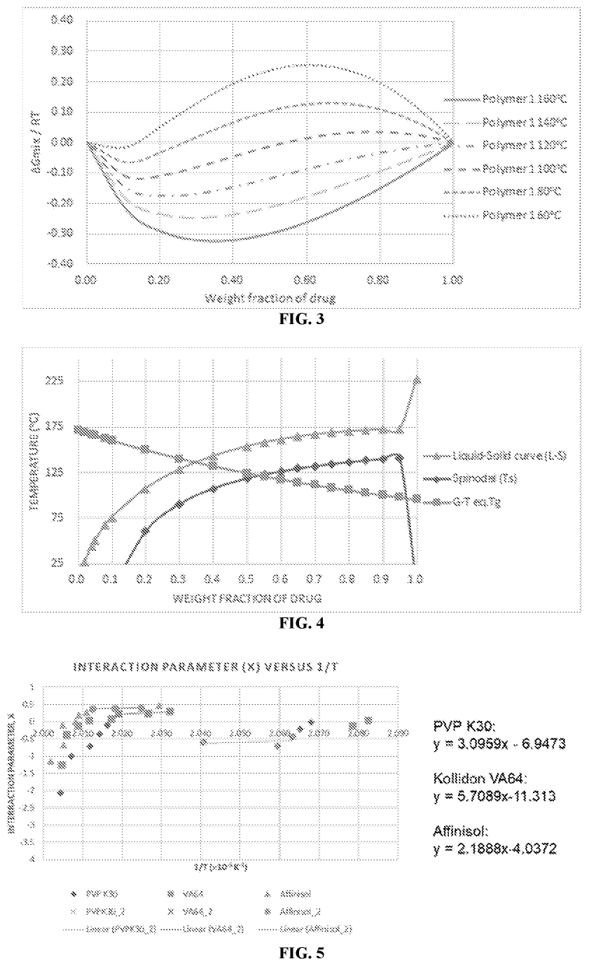

11 samples ranging from 45-95% by weight Atovaquone were mixed with PVP K30 including 4% Tween 20, 3% PEG 400 and 3% Benzyl Benzoate (i.e., A18 described in Table 2) so that melting point depression could be determined with DSC (DSC Q20, TA Instruments, New Castle, Del.) by weighing 5 mg samples, ramped at 10° C./min from 30-250° C. Results were combined with Flory-Huggins and used to calculate a chi value (interaction parameter). See FIGS. 3, 4, and 5.

### Example 4—Flory-Huggins Modeling for Atovaquone and Kollidone VA 64 Miscibility

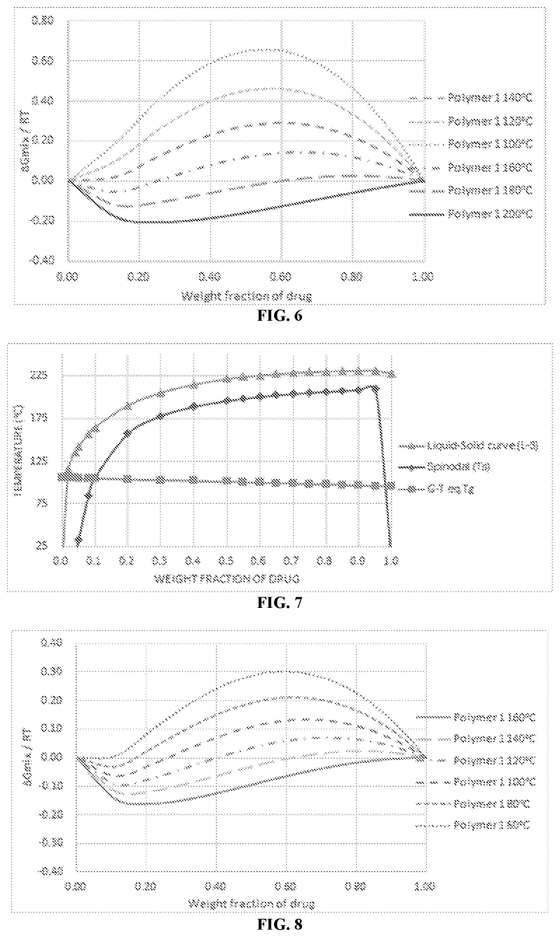

11 samples ranging from 45-95% by weight Atovaquone were mixed with Kollidon VA 64 including 4% Tween 20, 3% PEG 400 and 3% Benzyl Benzoate (i.e., A18 as described in Table 2) so that melting point depression could be determined with DSC (DSC Q20, TA Instruments, New Castle, Del.) by weighing 5 mg samples, ramped at 10° C./min from 30-250° C. Results were combined with Flory-Huggins and used to calculate a chi value resulting in a Gibbs free energy value for Kollidon VA-64 and ATQ. See FIGS. 5, 6, and 7.

### Example 5—Flory-Huggins Modeling for Atovaquone and Hypromellose Miscibility

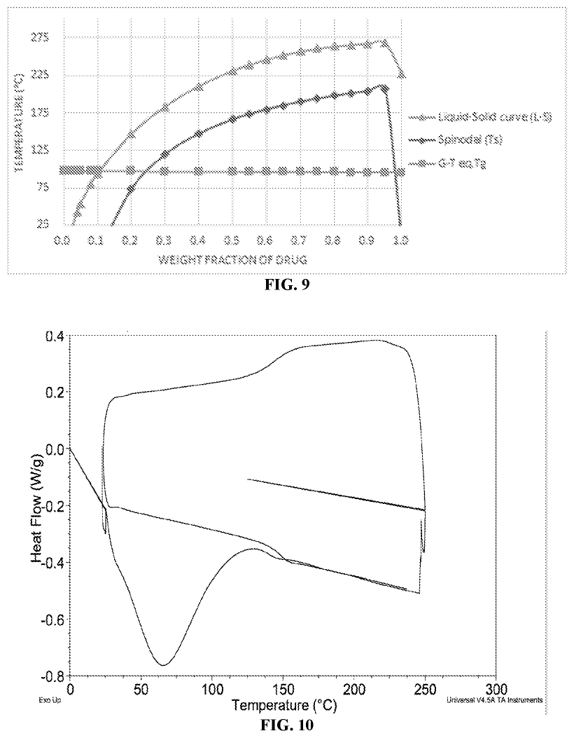

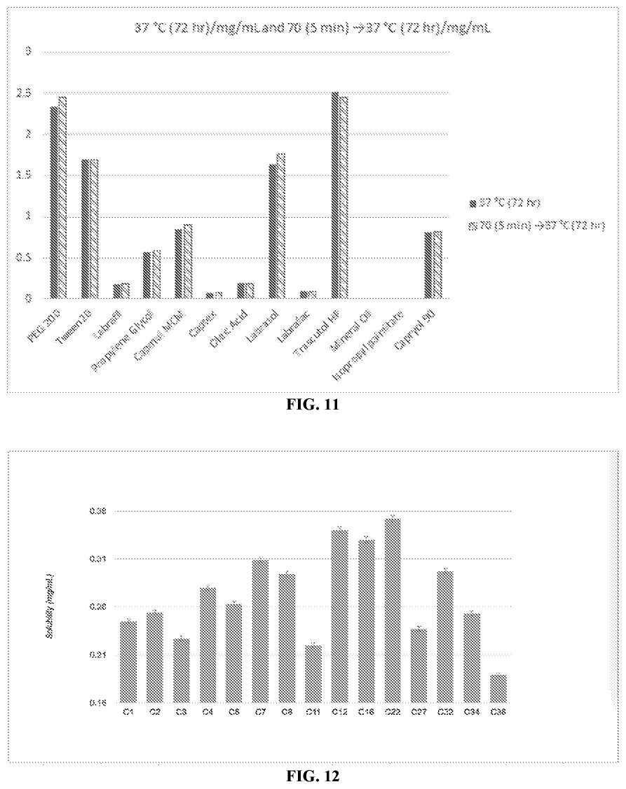

11 samples ranging from 45-95% by weight Atovaquone were mixed with Affinisol HME 15 LV including 4% Tween 20, 3% PEG 400 and 3% Benzyl Benzoate (i.e., A18 as described in Table 2) so that melting point depression could be determined with DSC (DSC Q20, TA Instruments, New Castle, Del.) by weighing 5 mg samples, ramped at 10° C./min from 30-250° C. Results were combined with Flory-Huggins and used to calculate a chi value resulting in a Gibbs free energy value for Affinisol and ATQ. See FIGS. 5, 8, and 9.

### Example 6—Hot Melt Extrusion of Drug, Spontaneous Emulsifying Component and Polymer

TABLE 4
Percent composition of each component in Formulation 1.

| Component | Percentage (%) | Amount |
|---|---|---|
| PEG 400 | 3 | 0.3 g |
| polysorbate 20 | 4 | 0.4 g |
| benzyl benzoate | 3 | 0.3 g |
| atovaquone | 20 | 2 g |
| PVP K30 | 70 | 7 g |
| Total | 100 | 10 g |

4% Tween 20, 3% PEG 400 and 3% Benzyl Benzoate were vortexed and then added to 20% Atovaquone and 70% PVP K30 which was then homogeneously mixed by mortar and pestle (Table 4). The premixed formulation was then added to a HAAKE Minilab II micro Compounder (Thermo Electron Corporation, Newington, N.H.) extruder with a screw speed of 150 RPM at 180° C. A dark red, transparent, low-viscosity melt was produced and remained clear after hardening from cooling and storage. The melt was milled to granules and passed through a 600 micron sieve that for future examples is referred to as Formulation 1.

### Example 7—Hot Melt Extrusion of Drug and Polymer

TABLE 5
Percent composition of components comprising Formulation 1a.

| Component | Percentages (%) | Amount |
|---|---|---|
| Atovaquone | 20 | 2 g |
| PVP K30 | 80 | 8 g |

20% Atovaquone and 80% PVP K30 was homogeneously mixed by mortar and pestle (Table 5). The premixed formulation was then added to a HAAKE Minilab 11 micro Compounder (Thermo Electron Corporation, Newington, N.H.) extruder with a screw speed of 150 RPM at 180° C. A black-red, non-transparent, low-viscosity melt was produced and remained clear after hardening from cooling and storage. The melt was milled and passed through a 600 micron sieve that for future examples is referred to as formulation 1a. DSC results shown in FIG. 10.

### Example 8—Hot Melt Extrusion of Formulation 1 Composition at 75 RPM and 120° C.

4% Tween 20, 3% PEG 400 and 3% Benzyl Benzoate were vortexed and then added to 20% Atovaquone and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 75 RPM at 120° C. A dark red-orange, transparent, low-viscosity melt was produced and turned a bright yellow color after hardening from cooling and storage. The melt was milled into granules and passed through a 600 micron sieve that for future examples is referred to as Formulation 2.

### Example 9—Hot Melt Extrusion of Formulation 1 Composition at 150 RPM and 80° C.

4% Tween 20, 3% PEG 400 and 3% Benzyl Benzoate were vortexed and then added to 20% Atovaquone and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 80° C. A light orange, non-transparent, low-viscosity melt was produced and turned to a bright yellow color after hardening from cooling and storage after 2 months. The melt was milled into granules and passed through a 600 micron sieve that for future examples is referred to as Formulation 3.

### Example 10—Hot Melt Extrusion of Formulation 1a Composition at 150 RPM and 160° C.

20% Atovaquone and 80% PVP K30 was homogeneously mixed by mortar and pestle. The premixed formulation was then added to a HAAKE Minilab 11 micro Compounder (Thermo Electron Corporation, Newington, N.H.) extruder with a screw speed of 150 RPM at 160° C. This composition caused the extruder to stop and therefore an extrudate was not produced.

### Example 11—Formulation Containing Only One Component of the Spontaneous Emulsifying Component: Tween 20

10% Tween 20 was added to 20% Atovaquone and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 180° C. A dark-red, transparent, low-viscosity melt was produced and remained transparent after hardening from cooling and storage. The melt was milled to granules and passed through a 600 micron sieve that for future examples is referred to as Formulation 4.

### Example 12—Formulation Containing Only One Component of the Spontaneous Emulsifying Component: PEG-400

10% PEG 400 was added to 20% Atovaquone and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 180° C. A dark black-red, transparent, low-viscosity melt was produced and remained transparent after hardening from cooling and storage. The melt was milled to granules and passed through a 600 micron sieve that for future examples is referred to as Formulation 4a.

### Example 13—Formulation Containing Only One Component of the Spontaneous Emulsifying Component: Benzyl Benzoate

10% Benzyl Benzoate was added to 20% Atovaquone and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 180° C. A dark black-red, transparent, low-viscosity melt was produced and remained transparent after hardening from cooling and storage. The melt was milled and passed through a 600 micron sieve that for future examples is referred to as Formulation 4b.

### Example 14—Formulation Composition at a Higher Drug Concentration

4% Tween 20, 3% PEG 400 and 3% Benzyl Benzoate were vortexed and then added to 30% Atovaquone and 60% PVP K30 which was then homogeneously mixed by mortar and pestle. The premixed formulation was then added to a HAAKE Minilab 11 micro Compounder (Thermo Electron Corporation, Newington, N.H.) extruder with a screw speed of 150 RPM at 180° C. A dark orange-red, transparent, low-viscosity melt was produced and remained transparent after hardening from cooling and storage. The melt was milled to granules and passed through a 600 micron sieve that for future examples is referred to as Formulation 5.

### Example 15—Formulation Using a 30% Drug Concentration without the Spontaneous Emulsifying Component

30% Atovaquone and 70% PVP K30 was homogeneously mixed by mortar and pestle. The premixed formulation was then added to a HAAKE Minilab II micro Compounder (Thermo Electron Corporation, Newington, N.H.) extruder with a screw speed of 150 RPM at 180° C. A dark black-red, transparent, low-viscosity melt was produced and remained transparent after hardening from cooling and storage. The melt was milled to granules and passed through a 600 micron sieve that for future examples is referred to as Formulation 5a.

### Example 16—Formulation Using a 60% Concentration of Drug with the Spontaneous Emulsifying Component

4% Tween 20, 3% PEG 400 and 3% Benzyl Benzoate were vortexed and then added to 60% Atovaquone and 30% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 180° C. A dark orange-red, transparent, low-viscosity melt was produced and remained (almost completely transparent after hardening from cooling and storage. The melt milled to granules and passed through a 600 micron sieve that for future examples is referred to as Formulation 6.

### Example 17—Formulation Using a 60% Drug Concentration without the Spontaneous Emulsifying Component

60% Atovaquone and 40% PVP K30 was homogeneously mixed by mortar and pestle. The premixed formulation was then added to a HAAKE Minilab II micro Compounder (Thermo Electron Corporation, Newington, N.H.) extruder with a screw speed of 150 RPM at 180° C. A dark black-red, transparent, low-viscosity melt was produced and remained transparent after hardening from cooling and storage. The melt was milled to granules and passed through a 600 micron sieve that for future examples is referred to as Formulation 6a.

### Example 18—Formulation Using a 20% Drug Concentration with the Spontaneous Emulsifying Component: Kollidon VA-64

4% Tween 20, 3% PEG 400 and 3% Benzyl Benzoate were vortexed and then added to 20% Atovaquone and 70% VA-64 which was then homogeneously mixed by mortar and pestle. The premixed formulation was then added to a HAAKE Minilab micro Compounder (Thermo Electron Corporation, Newington, N.H.) extruder with a screw speed of 150 RPM at 160° C. An amber colored, transparent, low-viscosity melt was produced and remained transparent (no spots) after hardening from cooling and storage. The melt was milled to granules and passed through a 600 micron sieve that for future examples is referred to as Formulation 7.

### Example 19—Formulation Using a 20% Drug Concentration without the Spontaneous Emulsifying Component: Kollidon VA-64

20% Atovaquone and 80% VA-64 was homogeneously mixed by mortar and pestle. The premixed formulation was then added to a HAAKE Minilab 11 micro Compounder (Thermo Electron Corporation, Newington, N.H.) extruder with a screw speed of 150 RPM at 160° C. A ambered colored, transparent, low-viscosity melt was produced and remained transparent (spots) after hardening from cooling and storage. The melt was milled to granules and passed through a 600 micron sieve that for future examples is referred to as Formulation 7a.

### Example 20—Formulation Using Mebendazole with the Spontaneous Emulsifying Component

4% Tween 20, 3% PEG 400 and 3% Benzyl Benzoate were vortexed and then added to 20% mebendazole (Molekula, Irvine, Calif.) and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 180° C. An off yellow color, transparent, low-viscosity melt was produced and remained transparent after hardening from cooling and storage. The melt was milled to granules and passed through a 600 micron sieve that for future examples is referred to as Formulation 8.

### Example 21—Formulation Using Mebendazole without the Spontaneous Emulsifying Component

20% Mebendazole and 80% PVP K30 was homogeneously mixed by mortar and pestle. The premixed formulation was then added to a HAAKE Minilab II micro Compounder (Thermo Electron Corporation, Newington, N.H.) extruder with a screw speed of 150 RPM at 180° C. Upon extrusion the extruder locked and a melt was not produced. The next temperature the HME was operable at was 220° C. at 150 RPM producing an orange-like color and regions of opacity. The melt at 220° C. was milled to granules and passed through a 600 micron sieve that for future examples is referred to as Formulation 8a.

### Example 22—Mebendazole Solubility in Pharmaceutical Excipients

Blends for pharmaceutical excipients were prepared as described in Table [symbol] components were also tested. Excess mebendazole (100 mg/mL) was added to each of the following excipients and blends: Benzyl Benzoate, Tween 20, Tween 80, PEG 200, PEG 400, Labrafac, Labrafil, Capryol, Capmul, Captex, Propylene Glycol and Isopropyl Palmitate. The mixtures were shaken at 100 RPM at 37° C. for 48 hours. Each individual sample was centrifuged at 3000 RPM for 10 minutes. The supernatant was then filtered through a 0.45 μm PTFE filter. Quantification of mebendazole content in the filtrate was performed using a Dionex HPLC (Thermo Fisher Scientific Inc., Waltham, Mass.) with an Inertsil® ODS-2 column (5 μm, 4.6×250 mm) with a mobile phase 0.1% ACN:0.05 M potassium phosphate dibasic in deionized water (60:40) at flow rate of 1 mL/min and a run time of 10 minutes. The results of the solubility study are shown in FIGS. 11 (for the individual pharmaceutical excipients) and 12 (for the blends of pharmaceutical excipients described in Table 6). Particle (droplet) size distribution data is also shown in Table 7 for use in constructing a ternary phase diagram in later example.

TABLE 6
Results of solubility study.

| | C1 | C2 | C3 | C4 | C5 | C7 | C8 | C11 | C12 | C16 | C22 | C27 | C32 | C34 | C36 |
|---|---|---|---|---|---|---|---|---|---|---|---|---|---|---|---|
| Tween 20 | 80 | 70 | 70 | 60 | 60 | 50 | 50 | 40 | 40 | 30 | 20 | 20 | 10 | 10 | 10 |
| PEG 200 | 10 | 20 | 10 | 30 | 20 | 40 | 30 | 50 | 40 | 60 | 70 | 20 | 50 | 30 | 10 |
| Capmul MCM NF | 10 | 10 | 20 | 10 | 20 | 10 | 20 | 10 | 20 | 10 | 10 | 60 | 40 | 60 | 80 |

TABLE 7
Size distribution data.

| | solubility | | | | | | |
|---|---|---|---|---|---|---|---|
| | 37° C. | Size distribution by volume | | | | | |
| sample | (72 hr) | Peak 1 | | Peak 2 | | Peak 3 | |
| name | mg/mL | size (d.nm) | % volume | size (d.nm) | % volume | size (d.nm) | % volume |
| C1 | 0.245 | 7.216 | 100 | 0 | 0 | 0 | 0 |
| C2 | 0.254 | 10.62 | 99.9 | 1493 | 0 | 4169 | 0 |
| C3 | 0.227 | 8.406 | 100 | 0 | 0 | 0 | 0 |
| C4 | 0.28 | 10.94 | 99.9 | 4446 | 0 | 1290 | 0 |
| C5 | 0.253 | 9.57 | 100 | 5246 | 0 | 0 | 0 |
| C7 | 0.309 | 9.485 | 100 | 4403 | 0 | 0 | 0 |
| C8 | 0.294 | 11.5 | 99.9 | 251.9 | 01 | 0 | 0 |
| C11 | 0.22 | 8.914 | 100 | 0 | 0 | 0 | 0 |
| C12 | 0.34 | 14.51 | 100 | 4684 | 0 | 0 | 0 |
| C16 | 0.33 | 13.18 | 99.9 | 545.3 | 0 | 5352 | 0 |
| C22 | 0.352 | 14.39 | 100 | 5210 | 0 | 0 | 0 |
| C27 | 0.237 | 87.14 | 100 | 0 | 0 | 0 | 0 |
| C32 | 0.297 | 119.6 | 19.4 | 24.03 | 80.6 | 0 | 0 |
| C34 | 0.253 | 71.8 | 69.8 | 641.4 | 30.2 | 0 | 0 |
| C36 | 0.189 | 312.4 | 45.4 | 4980 | 54.6 | 0 | 0 |

### Example 23—Formulation Using Mebendazole with a Spontaneous Emulsifying Component

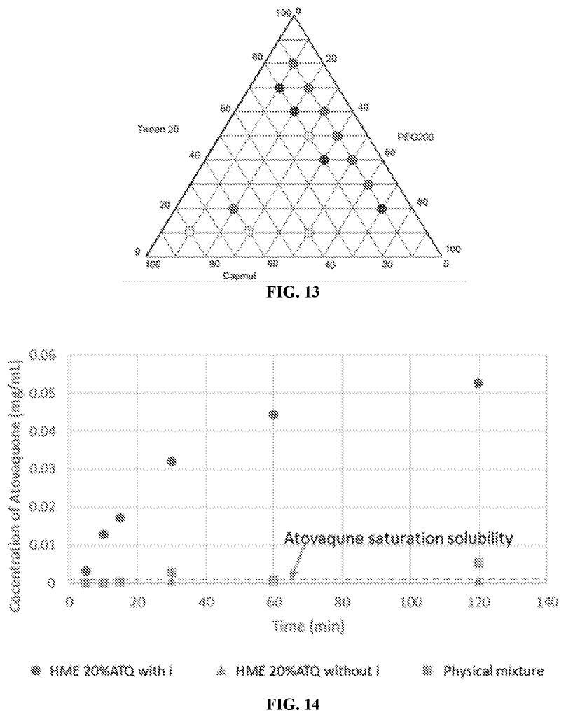

5% Tween 20, 4% PEG 400 and 1% Capmul MCM were vortexed and then added to 20% Mebendazole and 70% PVP K30 which was then homogeneously mixed by mortar and pestle. The premixed formulation was then added to a HAAKE Minilab II micro Compounder (Thermo Electron Corporation, Newington, N.H.) extruder with a screw speed of 150 RPM at 160° C. A, yellow-like color, opaque, low-viscosity melt was produced and remained opaque after hardening from cooling and storage. The melt was milled to granules and passed through a 600 micron sieve that for future examples is referred to as Formulation 9. Ternary phase diagram is shown in FIG. 13.

### Example 24—Formulation Using Mebendazole with a Spontaneous Emulsifying Component and Kollidon VA-64

4% Tween 20, 3% PEG 400 and 3% Benzyl Benzoate were vortexed and then added to 20% Mebendazole and 70% VA-64 which was then homogeneously mixed by mortar and pestle. The premixed formulation was then added to a HAAKE Minilab 11 micro Compounder (Thermo Electron Corporation, Newington, N.H.) extruder with a screw speed of 150 RPM at 160° C. A white color, transparent, low-viscosity melt was produced and remained transparent after hardening from cooling and storage. The melt was milled to granules and passed through a 600 micron sieve that for future examples is referred to as Formulation 10.

### Example 25—Formulation Using Mebendazole without a Spontaneous Emulsifying Component: PVP K30

20% Mebendazole and 80% PVP K30 was homogeneously mixed by mortar and pestle. The premixed formulation was then added to a HAAKE Minilab 11 micro Compounder (Thermo Electron Corporation, Newington, N.H.) extruder with a screw speed of 150 RPM at 160° C. An off-white color, opaque, low-viscosity melt was produced and remained opaque after hardening from cooling and storage. The melt was milled to granules and passed through a 600 micron sieve that for future examples is referred to as Formulation 11.

### Example 26—Formulation Using Mebendazole with a Spontaneous Emulsifying Component and Kollidon VA-64

5% Tween 20, 4% PEG 400 and 1% Capmul MCM were vortexed and then added to 20% Mebendazole and 70% VA-64 which was then homogeneously mixed by mortar and pestle. The premixed formulation was then added to a HAAKE Minilab II micro Compounder (Thermo Electron Corporation, Newington, N.H.) extruder with a screw speed of 150 RPM at 160° C. A white color, appeared bubbly and opaque, low-viscosity melt was produced and remained non-transparent after hardening from cooling and storage. The melt was milled to granules and passed through a 600 micron sieve that for future examples is referred to as Formulation 12.

### Example 27—Preparation of Physical Mixture of the Formulation

4% Tween 20, 3% PEG 400 and 3% Benzyl Benzoate were vortexed and then added to 20% Atovaquone and 70% PVP K30 which was then homogeneously mixed by mortar and pestle and referred to in future examples as Formulation 13.

### Example 28—Dispersibility Test in Water of a Tablet of Formulation 1

Granules of 300 mg of Formulation 1 were compressed into a tablet and when dissolution is performed at a paddle speed of 100 RPM at 37° C., the tablet immediately dispersed and became wetted. Atovaquone aliquots were directly sampled at time points of 5, 10, 15, 30, 60, and 120 minutes, centrifuged for 1 minute at 1000 RPM, diluted with ACN, assayed with the same HPLC method as described in Example 2 and shown in FIG. 14. Upon completion of the dispersibility test, the tablet had completely dissolved tablet.

### Example 29—Dispersibility Test in Water of a Tablet of Formulation 1a

Granules of 300 mg of formulation 1a was compressed into a tablet and when dissolution is performed at a paddle speed of 100 RPM at 37° C., the tablet did not immediately disperse or become available. Atovaquone aliquots were directly sampled at time points of 5, 10, 15, 30, 60, and 120 minutes, centrifuged for 1 minute at 1000 RPM, diluted with ACN, ran with the same HPLC method as Example 2 and shown in FIG. 14.

### Example 30—Dispersibility Test in Water of a Tablet of Formulation 13

Granules of 300 mg of formulation 13 was compressed into a tablet and when dissolution is performed at a paddle speed of 100 RPM at 37° C. the tablet did not immediately disperse or became rapidly available. Atovaquone aliquots were directly sampled to determine crystallinity and concentrations at time points of 5, 10, 15, 30, 60, 120 minutes, centrifuged for 1 minute at 1000 RPM, diluted with ACN, ran with the same HPLC method as Example 2 and shown in FIG. 14.

### Example 31—Dispersibility Test in Water of Granules of Formulation 7

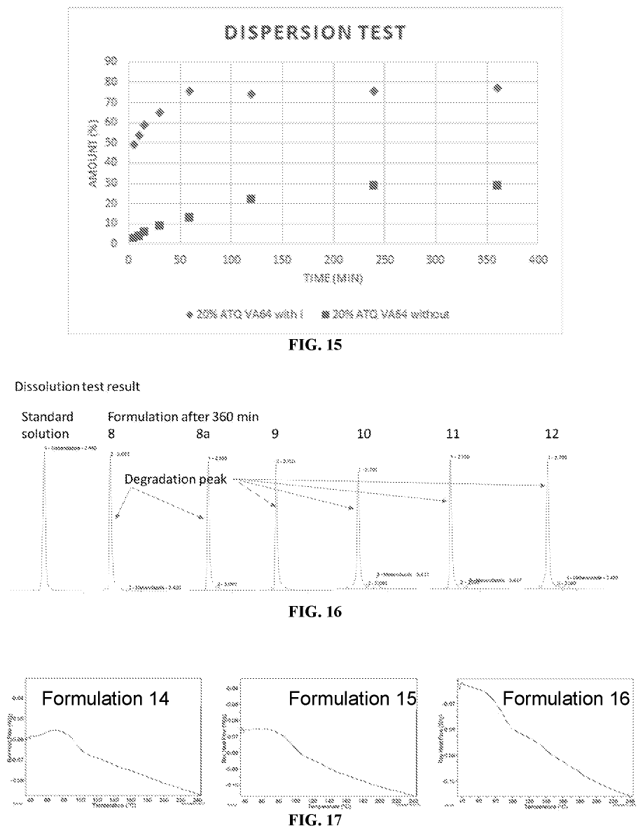

Granules of 300 mg of Formulation 7 were added to the dissolution. The dissolution was performed at a paddle speed of 100 RPM at 37° C. Atovaquone concentrations were directly sampled at time points of 5, 10, 15, 30, 60, 120, 240, and 360 minutes, centrifuged for 1 minute at 1000 RPM, diluted with ACN, ran with the same HPLC method as Example 2 and shown in FIG. 15.

### Example 32—Dispersibility Test in Water of Granules of Formulation 7a

Granules of 300 mg of Formulation 7a were added to the dissolution. The dissolution was performed at a paddle speed of 100 RPM at 37° C. Atovaquone concentrations were directly sampled at time points of 5, 10, 15, 30, 60, 120, 240, and 360 minutes, centrifuged for 1 minute at 1000 RPM, diluted with ACN, ran with the same HPLC method as Example 2 and shown in FIG. 15.

### Example 33—Dissolution Test for Formulation 8 PVP-K30 Containing 20% MBZ with a Spontaneous Emulsifying Component

Granules of 1000 mg of Formulation 8 were added to the dissolution. Dissolution was performed at a paddle speed of 100 RPM at 37° C. the product disperses. Mebendazole concentrations were directly sampled at time points of 5, 10, 15, 30, 60, 120, 240, and 360 minutes, filtered, diluted with ACN, assayed by HPLC, and shown in FIG. 16.

### Example 34—Formulation 8a Containing PVP-K30 and 20% MBZ without a Spontaneous Emulsifying Component

Granules of 1000 mg of Formulation 8a were added to the dissolution. Dissolution was performed at a paddle speed of 100 RPM at 37° C. Mebendazole concentrations were directly sampled at time points of 5, 10, 15, 30, 60, 120, 240, and 360 minutes, filtered, diluted with ACN, assayed by HPLC, and shown in FIG. 16.

### Example 35—Formulation 9 Containing PVP-K30 and MBZ with a Spontaneous Emulsifying Component

Granules of 1000 mg of Formulation 9 were added to the dissolution. Dissolution was performed at a paddle speed of 100 RPM at 37° C. Mebendazole concentrations were directly sampled at time points of 5, 10, 15, 30, 60, 120, 240, and 360 minutes, filtered, diluted with ACN, assayed by HPLC, and shown in FIG. 16.

### Example 36—Formulation 10 Containing Kollidon VA-64 with a Spontaneous Emulsifying Component

Granules of 1000 mg of Formulation 10 were added to the dissolution. Dissolution was performed at a paddle speed of 100 RPM at 37° C. Mebendazole concentrations were directly sampled at time points of 5, 10, 15, 30, 60, 120, 240, and 360 minutes, filtered, diluted with ACN, assayed by HPLC, and shown in FIG. 16.

### Example 37—Formulation 11 Containing Kolidon VA-64 and Mebendazole without a Spontaneous Emulsifying Component

Granules of 1000 mg of Formulation 11 were added to the dissolution. Dissolution was performed at a paddle speed of 100 RPM at 37° C. Mebendazole concentrations were directly sampled at time points of 5, 10, 15, 30, 60, 120, 240, and 360 minutes, filtered, diluted with ACN, assayed by HPLC, and shown in FIG. 16.

### Example 38—Formulation 12 Containing Kollidon VA-64 and Mebendazole with a Spontaneous Emulsifying Component

Granules of 1000 mg of Formulation 12 were added to the dissolution. Dissolution was performed at a paddle speed of 100 RPM at 37° C. Mebendazole concentrations were directly sampled at time points of 5, 10, 15, 30, 60, 120, 240, and 360 minutes, filtered, diluted with ACN, assayed by HPLC, and shown in FIG. 16.

### Example 39—Dispersibility in Water for Granules of Formulation 1

Granules of 300 mg of Formulation 1 were added to the dissolution bath. The dispersability test was performed as described in Example 28. Atovaquone aliquots were directly sampled at time points of 5, 10, 15, 30, 60, 120, 240, and 360 minutes. Granules of Formulation 1 were wetted and dispersed immediately upon contact with the water media.

### Example 40—Dispersibility in Water for Granules of Formulation 1a

Granules of 300 mg of Formulation 1 were added to the dissolution bath. The dispersability test was performed as described in Example 28. Atovaquone aliquots were directly sampled at time points of 5, 10, 15, 30, 60, 120, 240, and 360 minutes. Granules of Formulation 1a were not wetted nor dispersed upon contact with the water media until after about 1 hour.

### Example 41—Dispersibility in Water for Granules of Formulation 13

Granules of 300 mg of Formulation 13 were added to the dissolution bath. The dispersability test was performed as described in Example 28. Atovaquone aliquots were directly sampled at time points of 5, 10, 15, 30, 60, 120, 240, and 360 minutes. Granules of Formulation 13 were not wetted nor dispersed upon contact with the water media until after about 1 hour.

### Example 42—Dispersibility in Water for Granules of Formulation 4

Granules of 300 mg of Formulation 4 were added to the dissolution bath. The dispersability test was performed as described in Example 28. Atovaquone aliquots were directly sampled at time points of 5, 10, 15, 30, 60, 120, 240, and 360 minutes. Granules of Formulation 4 were immediately wetted and dispersed upon contact with the water media but presence of crystals of drug occurred within about 1 hour.

### Example 43—Dispersibility in Water for Granules of Formulation 4a

Granules of 300 mg of Formulation 4a were added to the dissolution bath. The dispersability test was performed as described in Example 28. Atovaquone aliquots were directly sampled at time points of 5, 10, 15, 30, 60, 120, 240, and 360 minutes. Granules of Formulation 4a were not immediately wetted and dispersed upon contact with the water media and presence of crystals of drug occurred within about 1 hour.

### Example 44—Dispersibility in Water for Granules of Formulation 4b

Granules of 300 mg of Formulation 4b were added to the dissolution bath. The dispersability test was performed as described in Example 28. Atovaquone aliquots were directly sampled at time points of 5, 10, 15, 30, 60, 120, 240, and 360 minutes. Granules of Formulation 4b were not immediately dispersed and presence of crystallinity was not observed.

### Example 45—Dispersibility in Water for Granules of Formulation 5

Granules of 300 mg of Formulation 5 were added to the dissolution bath. The dispersability test was performed as described in Example 28. Atovaquone aliquots were directly sampled at time points of 5, 10, 15, 30, 60, 120, 240, and 360 minutes. Granules of Formulation 5 were immediately dispersed and presence of crystallinity was not observed.

### Example 46—Dispersibility in Water for Granules of Formulation 5a

Granules of 300 mg of Formulation 5a were added to the dissolution bath. The dispersability test was performed as described in Example 28. Atovaquone aliquots were directly sampled at time points of 5, 10, 15, 30, 60, 120, 240, and 360 minutes. Granules of Formulation 5a were not immediately dispersed and presence of crystallinity was observed.

### Example 47—Preparation and Solubility Properties of Formulations 14-17

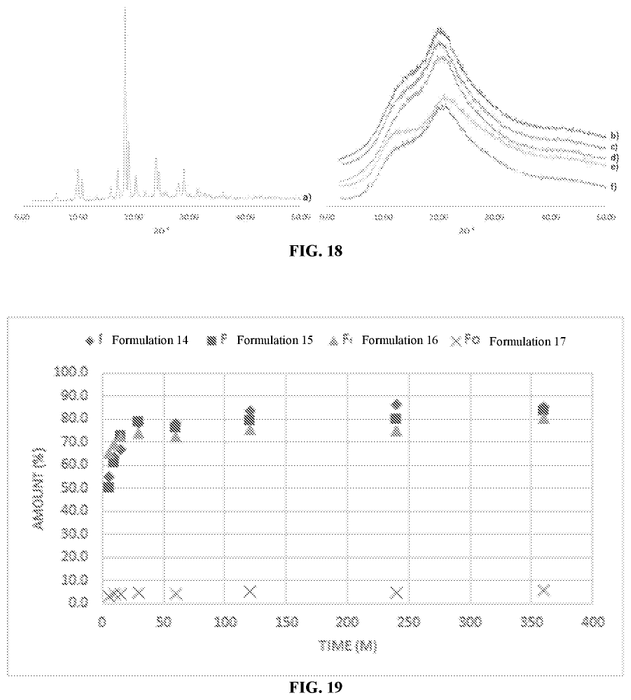

Formulations 14-17 (shown in Table 8) were prepared as follows. The ingredients of the composition were blended together and then thermally processed using a Mini Haake hot melt extruder. The conditions were: 180° C., 150 rpm screw speed; batch size 10 g. The extrudate was cooled to room temperature and then mechanically milled using a grinder to form granules. The granules were passed through a 600 micron sieve. Modulated differential scanning calorimetry data for formulations 14-16 is shown in FIG. 17. Powder X-Ray diffraction data for Formulations 1, 14-17 and bulk atovaquone are shown in FIG. 18. Dispersibility test for granules of formulations 14-17 (as shown in Table 8) is shown in FIG. 19. The dispersion test was conducted using USP Apparatus 2 (paddle) and the following test conditions: deionized water (900 mL); Temperature 37° C.; paddle speed 100 rpm; sample times as indicated on the X-axis in FIG. 19. The amount of atovaquone from the granules remaining dispersed following centrifugation (1000 rpm for 1 min) was determined by HPLC.

TABLE 8
Percent composition values for
components comprised in formulations 14-17

| | Formulation | Formulation | Formulation | Formulation |
|---|---|---|---|---|
| Component | 14 (% w/w) | 15 (% w/w) | 16 (% w/w) | 17 (% w/w) |
| Atovaquone | 20 | 20 | 20 | 20 |
| PVP K30 | 70 | 70 | 70 | 80 |
| Capmul | 2.5 | 2.5 | 0.5 | — |
| MCM EP/NF | | | | |
| (Glyceryl | | | | |
| Caprylate/ | | | | |
| Caprate) | | | | |
| Captex 300 | 2.5 | 2.5 | 0.5 | — |
| EP/NF | | | | |
| (Glyceryl | | | | |
| Tricaprylate/ | | | | |
| Tricaprate) | | | | |
| Polysorbate 80 | 5 | — | — | — |
| Cremophor | — | 5 | — | — |
| RH40 | | | | |
| Labrasol | — | — | 9 | — |

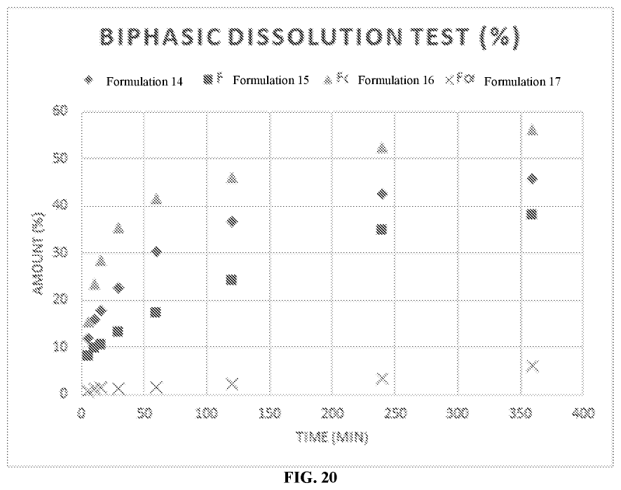

Biphasic dissolution test data for formulations 14-17 was obtained. Drug release of atovaquone from granules of formulations 14-17 (as shown in Table 4) into the aqueous dissolution media (either as a small particle or dissolved) and dissolve into the octanol phase was determined. Aliquots of 300 mg of each formulation was added to 700 mL of water phase. Subsequently, 200 mL of octanol was added to create the biphasic dissolution test media. The test was performed at a paddle speed of 200 RPM at 37° C. The granules of formulations 14-16 immediately wet and disperse. Atovaquone concentrations were directly sampled from the octanol phase at time points shown in FIG. 20, filtered using a 0.22 micron PTFE filter, diluted with ACN, and assayed by HPLC.

### Example 48—Preparation of Formulation 18

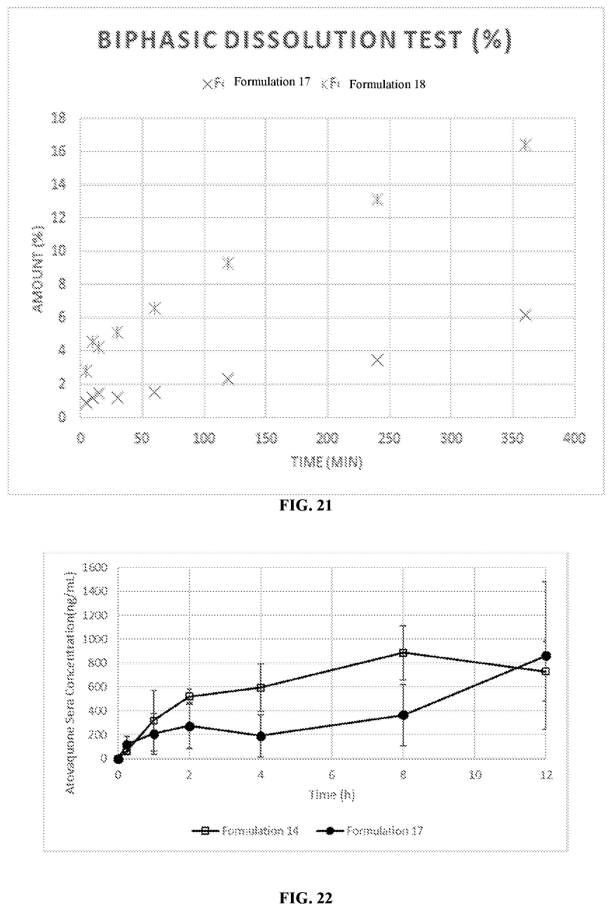

Formulation 18 (also the same composition as Formulation 1) was prepared as follows. Atovaquone, PVP K30, benzyl benzoate, Tween 20 and PEG 400 (20:70:3:4:3 w/w) were blended together and then thermally processed using a Mini Haake hot melt extruder at 180° C., 150 rpm screw speed, and a batch size of 10 g. The extrudate was cooled to room temperature and then milled using a grinder to form granules. The granules were passed through a 600 micron sieve. The biphasic dissolution test was performed as described in Example 47 and the results are shown in FIG. 21 along with a comparison to Formulation 17 from Example 47.

### Example 49—Effect of Formulation 14 on the Pharmacokinetics of Atovaquone in Mice

Size M capsules were loaded with 3 mg of either Formulation 14 or Formulation 17. Capsules were dosed to C57Cl/6 mice (3 mice per formulation, per time point) and mice were sacrificed at 0.25, 1, 2, 4, 8 and 12 hours for harvesting. Their serum was collected and immediately frozen at −80° C. until analysis. Analysis was performed by high performance liquid chromatography with a Microsorb-MV® 100 (5 micron, 4.6×150 mm) column and mobile phase consisting of 0.1% TFA in acetonitrile: 0.1% TFA in deionized water (80:20) at a flow rate of 1 mL/min. Atovaquone was measured by UV absorbance at a wavelength of 251 nm. Atovaquone concentration in the mice sera over time is illustrated in FIG. 22. Formulation 14 resulted in a significantly higher AUC₀₋₁₂ₕ (p-value <0.01) than Formulation 17: 7800.7±158.6 ng·h/mL compared to 4422.2±592.6, respectively. (Takabe et al., 2018)

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 8,486,423
U.S. Pat. No. 9,339,440
Bansal et al., AAPS PharmSciTech, 17(2): p. 318-327, 2016.
Crowley et al., Drug Dev. Ind. Pharm., 33(10):909-926, 2007.
DiNunzio et al., Drug Development and Industrial Pharmacy, 36(9):1064-78, 2010d.
DiNunzio et al., European Journal of Pharmaceutical Sciences, 40(3):179-87, 2010c.
DiNunzio et al., European Journal of Pharmaceutics and Biopharmaceutics, 74(2):340-51, 2010a.
DiNunzio et al., Journal of Pharmaceutical Sciences, 99(3):1239-53, 2010b.
Friesen et al., Molecular Pharmaceutics, 5(6):1003-1019, 2008.
Huang et al., AAPS PharmSciTech, 17(1): p. 106-119, 2016.
Hughey et al., AAPS PharmSciTech, 11(2):760-74, 2010.
Hughey et al., International Journal of Pharmaceutics, 419(1):222-30, 2011.
Keen et al., Int. J. Pharm., 450(1-2):185-196, 2013.
LaFountaine et al., AAPS PharmSciTech, 17(1):43-, 2016a.
LaFountaine et al., European Journal of Pharmaceutics and Biopharmaceutics, 101:72-81, 2016.
Lang et al., Drug Dev. Ind. Pharm., 40(9):1133-1155, 2014.
Lin and Huang, International Journal of Pharmaceutics, 399(1): p. 109-115, 2010.
Lu et al., Pharmaceutical Development and Technology, 20(1): p. 105-117, 2015.
Maleki et al., J. Controlled Release, 262:329-347, 2017.
Marsac et al., Journal of Pharmaceutical Sciences, 99(1): p. 169-185, 2010.
Marsac et al., Pharmaceutical Research, 23(10): p. 2417, 2006.
Marsac et al., Pharmaceutical Research, 26(1): p. 139, 2008.
Prasad et al., “Emerging Technologies to Increase the Bioavailability of Poorly Water-Soluble Drugs,” In: Williams III RO, Watts AB, Miller DA, editors. Formulating Poorly Water Soluble Drugs. Second ed. Cham, Switzerland: Springer International; p. 691-740, 2016.
Purohit and Taylor, Molecular Pharmaceutics, 12(5): p. 1623-1635, 2015.
Qian et al., Journal of Pharmaceutical Sciences, 99(7): p. 2941-2947, 2010.
Repka et al., Drug Dev. Ind. Pharm., 33(10):1043-1057, 2007.
Sayed et al., AAPS PharmSciTech, 18(5):1507-1525, 2017.
Takabe et al., Pharmaceutics, 10(2):60, 2018
Tian et al., Drug Discovery Today, 13:606-612, 2008.
Tian et al., Molecular Pharmaceutics, 10(1): p. 236-248, 2013.
Vynckier et al., J. Pharm. Pharmacol., 66(2):167-179, 2014.
Zhao et al., Journal of Pharmaceutical Sciences, 100(8): p. 3196-3207, 2011.

What is claimed is:

1. A pharmaceutical composition comprising:
(A) a therapeutic agent;
(B) a pharmaceutically acceptable polymer; and
(C) a spontaneously emulsifying component, wherein the emulsifying component comprises:
(i) a lipid, solvent, or oil; and
(ii) at least 1% w/w relative to the weight of the composition of a surfactant or hydrophilic solvent.

2. The pharmaceutical composition of claim 1, wherein the pharmaceutical composition is prepared using a thermal process or a fusion-based high energy mixing process that does not require external heat input

3. The pharmaceutical composition of claim 2, wherein the thermal process is hot melt extrusion.

4. The pharmaceutical composition of claim 2, wherein the thermal process is a hot melt granulation process.

5. The pharmaceutical composition of either claim 3 or claim 4, wherein the thermal process is carried out at a temperature below the melting point of the therapeutic agent.

6. The pharmaceutical composition of either claim 3 or claim 4, wherein the thermal process is carried out at a temperature below the decomposition temperature of the therapeutic agent as measured by thermogravimetric analysis.

7. The pharmaceutical composition of claim 2, wherein the pharmaceutical composition has been processed through a fusion-based high energy mixing process that does not require external heat input that results in an increase in temperature.

8. The pharmaceutical composition of claim 7, wherein the increase in temperature results from frictional or shear energy

9. The pharmaceutical composition of claim 7, wherein the pharmaceutical composition has been processed by a thermokinetic mixing process.

10. The pharmaceutical composition according to any one of claims 1-9, wherein the therapeutic agent has a solubility in water of less than 5 mg/mL.

11. The pharmaceutical composition of claim 10, wherein the therapeutic agent is a Biopharmaceutics Classification System Class II or IV compound.

12. The pharmaceutical composition according to any one of claims 1-11, wherein the therapeutic agent is known to undergo thermal degradation.

13. The pharmaceutical composition of claim 12, wherein the therapeutic agent is known to undergo thermal degradation at a temperature greater than 80° C.

14. The pharmaceutical composition according to any one of claims 1-13, wherein the therapeutic agent is substantially present as an amorphous form or as a molecular solution.

15. The pharmaceutical composition of claim 14, wherein the therapeutic agent is essentially present as the amorphous form or as a molecular solution.

16. The pharmaceutical composition according to any one of claims 1-15, wherein the therapeutic agent is not albendazole, benomyl, benzimidazole fungicide, carbendazim, ciclobendazole, fenbendazole, flubendazole, mebendazole, nocodazole, oxfendazole and oxibendazole.

17. The pharmaceutical composition according to any one of claims 1-16, wherein the pharmaceutically acceptable polymer is a cellulosic polymer.

18. The pharmaceutical composition of claim 17, wherein the pharmaceutically acceptable polymer is a neutral cellulosic polymer.

19. The pharmaceutical composition of claim 17, wherein the pharmaceutically acceptable polymer is an ionizable cellulosic polymer.

20. The pharmaceutical composition according to any one of claims 1-16, wherein the pharmaceutically acceptable polymer is a non-cellulosic polymer.

21. The pharmaceutical composition of claim 20, wherein the pharmaceutically acceptable polymer is a neutral non-cellulosic polymer.

22. The pharmaceutical composition of claim 20, wherein the pharmaceutically acceptable polymer is an ionizable non-cellulosic polymer.

23. The pharmaceutical composition of claim 22, wherein the pharmaceutically acceptable polymer is a polymethacrylate or polyacrylate functionalized with a carboxylic acid group.

24. The pharmaceutical composition according to any one of claims 1-23, wherein one or more of lipids, solvent, or oils is in the liquid phase.

25. The pharmaceutical composition according to any one of claims 1-24, wherein the emulsifying composition comprises a lipid or oil.

26. The pharmaceutical composition of claim 25, wherein the lipid or oil is an ester of a fatty acid.

27. The pharmaceutical composition of claim 26, wherein the lipid or oil is a glyceride ester of one, two, or three fatty acids.

28. The pharmaceutical composition of either claim 26 or claim 27, wherein the fatty acids is medium chain fatty acids.

29. The pharmaceutical composition of claim 28, wherein the lipid or oil is Capmul®.

30. The pharmaceutical composition according to any one of claims 1-24, wherein the spontaneously emulsifying composition comprises a solvent.

31. The pharmaceutical composition of claim 30, wherein the solvent is a hydrophobic solvent.

32. The pharmaceutical composition of either claim 30 or claim 31, wherein the solvent contains one or more aromatic groups.

33. The pharmaceutical composition according to any one of claims 30-32, wherein the solvent is benzyl benzoate.

34. The pharmaceutical composition according to any one of claims 1-33, wherein the spontaneously emulsifying composition comprises a surfactant or hydrophilic solvent that contains one or more polyethylene glycol or polypropylene glycol repeating units.

35. The pharmaceutical composition of claim 34, wherein the hydrophilic solvent is a PEG polymer.

36. The pharmaceutical composition of claim 35, wherein the hydrophilic solvent is a PEG polymer with a molecular weight from 100 Daltons to 2000 Daltons.

37. The pharmaceutical composition of claim 36, wherein the hydrophilic solvent is PEG 200 or PEG 400.

38. The pharmaceutical composition according to any one of claims 1-37, wherein the pharmaceutical composition comprises a first surfactant.

39. The pharmaceutical composition of claim 38, wherein the first surfactant is polyethoxylated castor oil.

40. The pharmaceutical composition of claim 39, wherein the first surfactant is Cremophor EL.

41. The pharmaceutical composition according to any one of claims 1-40, wherein the pharmaceutical composition further comprises a second surfactant.

42. The pharmaceutical composition of claim 41, wherein the second surfactant is a compound with a hydrophobic component and a PEG or polypropylene glycol component.

43. The pharmaceutical composition of claim 42, wherein the hydrophobic component is a fatty acid.

44. The pharmaceutical composition of either claim 42 or claim 43, wherein the PEG or polypropylene glycol component is a PEGylated polysorbate.

45. The pharmaceutical composition according to any one of claims 41-44, wherein the second surfactant is Tween®.

46. The pharmaceutical composition according to any one of claims 1-45, wherein the therapeutic agent comprises from about 10% w/w to about 60% w/w of the total weight of composition.

47. The pharmaceutical composition of claim 46, wherein the therapeutic agent comprises from about 20% w/w to about 50% w/w.

48. The pharmaceutical composition of claim 47, wherein the therapeutic agent comprises from about 20% w/w to about 40% w/w.

49. The pharmaceutical composition according to any one of claims 1-48, wherein the therapeutic agent is present at a concentration greater than the solubility of the therapeutic agent in the pharmaceutically acceptable polymer alone.

50. The pharmaceutical composition according to any one of claims 1-48, wherein the therapeutic agent is present at a concentration greater than the solubility of the therapeutic agent in the spontaneously emulsifying component alone.

51. The pharmaceutical composition of either claim 49 or claim 50, wherein the therapeutic agent is present at a concentration greater than the solubility of the therapeutic agent in either the pharmaceutically acceptable polymer or the spontaneously emulsifying component alone.

52. The pharmaceutical composition according to any one of claims 49-51, wherein the therapeutic agent is present at a concentration greater than the solubility of the therapeutic agent in the pharmaceutically acceptable polymer or the spontaneously emulsifying component combined.

53. The pharmaceutical composition according to any one of claims 1-52, wherein the therapeutic agent is formulated such that at least 10% of the therapeutic agent is present in the undissolved form when added to or diluted in physiological fluid or water.

54. The pharmaceutical composition of claim 53, wherein at least 50% of the therapeutic agent is present in the undissolved form when added to or diluted in physiological fluid or water.

55. The pharmaceutical composition according to any one of claims 1-54, wherein the pharmaceutically acceptable polymer comprises from about 20% w/w to about 80% w/w of the total weight of the composition.

56. The pharmaceutical composition of claim 55, wherein the pharmaceutically acceptable polymer comprises from about 40% w/w to about 80% w/w.

57. The pharmaceutical composition of claim 56, wherein the pharmaceutically acceptable polymer comprises from about 50% w/w to about 80% w/w.

58. The pharmaceutical composition according to any one of claims 1-57, wherein the lipid, oil, or solvent comprises from about 0.25% w/w to about 10% w/w of the total weight of composition.

59. The pharmaceutical composition of claim 58, wherein the lipid, oil, or solvent comprises from about 0.5% w/w to about 5% w/w.

60. The pharmaceutical composition of claim 59, wherein the lipid, oil, or solvent comprises from about 1% w/w to about 3% w/w.

61. The pharmaceutical composition according to any one of claims 1-60, wherein the surfactant comprises from 2% w/w to about 10% w/w of the total weight of composition.

62. The pharmaceutical composition of claim 61, wherein the surfactant comprises from 2% w/w to about 6% w/w.

63. The pharmaceutical composition of claim 62, wherein the surfactant comprises from 3% w/w to about 5% w/w.

64. The pharmaceutical composition according to any one of claims 1-60, wherein the hydrophilic solvent comprises from 2% w/w to about 10% w/w of the total weight of composition.

65. The pharmaceutical composition of claim 64, wherein the hydrophilic solvent comprises from 2% w/w to about 6% w/w.

66. The pharmaceutical composition of claim 65, wherein the hydrophilic solvent comprises from 3% w/w to about 5% w/w.

67. The pharmaceutical composition according to any one of claims 1-66, wherein the second surfactant comprises from 2% w/w to about 10% w/w of the total weight of composition.

68. The pharmaceutical composition of claim 67, wherein the second surfactant comprises from 2% w/w to about 6% w/w.

69. The pharmaceutical composition of claim 68, wherein the second surfactant comprises from 3% w/w to about 5% w/w.

70. The pharmaceutical composition according to any one of claims 1-69, wherein the therapeutic agent is substantially free of any crystallinity.

71. The pharmaceutical composition of claim 70, wherein the therapeutic agent is essentially free of any crystallinity.

72. The pharmaceutical composition according to any one of claims 1-71, wherein the pharmaceutical composition exhibits a Flory-Huggins interaction parameter (χ) of greater than 0.25 as determined by differential scanning calorimetry (DSC).

73. The pharmaceutical composition of claim 72, wherein the Flory-Huggins interaction parameter is greater than 1.

74. The pharmaceutical composition according to any one of claims 1-71, wherein the pharmaceutical composition exhibits a negative Flory-Huggins interaction parameter (χ) as determined by differential scanning calorimetry (DSC).

75. The pharmaceutical composition according to any one of claims 1-74, wherein the composition is substantially free of fatty acids.

76. The pharmaceutical composition of claim 75, wherein the composition is essentially free of any fatty acids.

77. The pharmaceutical composition according to any one of claims 1-76, wherein the pharmaceutical composition further comprises an excipient.

78. The pharmaceutical composition of claim 77, wherein the excipient is a lubricant, disintegrant, binder, filler, surfactant, or any combination thereof

79. A method of preparing a pharmaceutical composition comprising:
(A) obtaining a composition comprising
(1) a therapeutic agent;
(2) a pharmaceutically acceptable polymer; and
(3) a spontaneously emulsifying component, wherein the spontaneously emulsifying component comprises:
(i) a lipid, solvent, or oil; and
(ii) at least 1% w/w relative to the weight of the composition of a surfactant or hydrophilic solvent; and
(B) heating the composition through a thermal process or a fusion-based high energy mixing process that does not require external heat input.

80. The method of claim 79, wherein the composition is obtained by adding the therapeutic agent, a pharmaceutically acceptable polymer, and the spontaneously emulsifying component.

81. The method of claim 80, wherein the composition is obtained by admixing the therapeutic agent, a pharmaceutically acceptable polymer, and the spontaneously emulsifying component.

82. The method of claim 79, wherein the composition is obtained from a third party.

83. The method according to any one of claims 79-82, wherein the lipid, oil, or solvent and the surfactant or hydrophilic solvent are added together to form the spontaneously emulsifying component before addition to the therapeutic agent and the pharmaceutically acceptable polymer.

84. The method of claim 83, wherein the spontaneously emulsifying component is mixed before addition to the therapeutic agent and the pharmaceutically acceptable polymer.

85. The method of claim 84, wherein the spontaneously emulsifying component is mixed by vortexing, blending, stirring, kneading, or homogenization.

86. The method of claim 84, wherein the spontaneously emulsifying component is added via a side port in the extruder during extrusion.

87. The method according to any one of claims 79-82, wherein the lipid, oil, or solvent and the surfactant or hydrophilic solvent is added independently to the pharmaceutically acceptable polymer and the therapeutic agent.

88. The method according to any one of claims 79-87, wherein the composition is mixed before heating.

89. The method of claim 88, wherein the composition is mixed using a homogenizer, a mixer, a vortexer, mortar and pestle, or a kneader.

90. The method according to any one of claims 79-89, wherein the composition is not mixed before heating.

91. The method according to any one of claims 79-90, wherein the thermal process is hot melt extrusion.

92. The method according to any one of claims 79-90, wherein the thermal process is a hot melt granulation process.

93. The method according to any one of claims 79-92, wherein the thermal process is carried out at a temperature below the melting point of the therapeutic agent.

94. The method according to any one of claims 79-93, wherein the thermal process is carried out at a temperature below the decomposition temperature of the therapeutic agent as measured by thermogravimetric analysis.

95. The method according to any one of claims 79-90, wherein the composition is processed through a fusion-based high energy mixing process that does not require external heat input that results in an increase in temperature.

96. The method of claim 95, wherein the increase in temperature results from frictional or shear energy

97. The method of claim 95, wherein the composition has been processed by a thermokinetic mixing process.

98. The method according to any one of claims 79-97, wherein the temperature of the thermal process is below the temperature required to obtain no crystallinity when formulated with the pharmaceutically acceptable polymer.

99. The method according to any one of claims 79-98, wherein the therapeutic agent comprises from about 10% w/w to about 60% w/w of the total weight of composition.

100. The method of claim 99, wherein the therapeutic agent comprises from about 20% w/w to about 50% w/w.

101. The method of claim 100, wherein the therapeutic agent comprises from about 20% w/w to about 40% w/w.

102. The method according to any one of claims 79-101, wherein the therapeutic agent is present at a concentration greater than the solubility of the therapeutic agent in the pharmaceutically acceptable polymer alone.

103. The method according to any one of claims 79-101, wherein the therapeutic agent is present at a concentration greater than the solubility of the therapeutic agent in the spontaneously emulsifying component alone.

104. The method of either claim 102 or claim 103, wherein the therapeutic agent is present at a concentration greater than the solubility of the therapeutic agent in either the pharmaceutically acceptable polymer or the spontaneously emulsifying component alone.

105. The method according to any one of claims 101-104, wherein the therapeutic agent is present at a concentration greater than the solubility of the therapeutic agent in either the pharmaceutically acceptable polymer or the spontaneously emulsifying component combined.

106. The method according to any one of claims 79-105, wherein the therapeutic agent is formulated such that at least 10% of the therapeutic agent is present in the undissolved form when added to or diluted in physiological fluid or water.

107. The method of claim 106, wherein at least 50% of the therapeutic agent is present in the undissolved form when added to or diluted in physiological fluid or water.

108. The method according to any one of claims 79-107, wherein the pharmaceutically acceptable polymer comprises from about 20% w/w to about 80% w/w of the total weight of the composition.

109. The method of claim 108, wherein the pharmaceutically acceptable polymer comprises from about 40% w/w to about 80% w/w.

110. The method of claim 109, wherein the pharmaceutically acceptable polymer comprises from about 50% w/w to about 80% w/w.

111. The method according to any one of claims 79-110, wherein the lipid, oil, or solvent comprises from about 0.25% w/w to about 10% w/w of the total weight of composition.

112. The method of claim 111, wherein the lipid, oil, or solvent comprises from about 0.5% w/w to about 5% w/w.

113. The method of claim 112, wherein the lipid, oil, or solvent comprises from about 1% w/w to about 3% w/w.

114. The method according to any one of claims 79-113, wherein the surfactant comprises from 2% w/w to about 10% w/w of the total weight of composition.

115. The method of claim 114, wherein the surfactant comprises from 2% w/w to about 6% w/w.

116. The method of claim 115, wherein the surfactant comprises from 3% w/w to about 5% w/w.

117. The method according to any one of claims 79-116, wherein the hydrophilic solvent comprises from 2% w/w to about 10% w/w of the total weight of composition.

118. The method of claim 117, wherein the hydrophilic solvent comprises from 2% w/w to about 6% w/w.

119. The method of claim 118, wherein the hydrophilic solvent comprises from 3% w/w to about 5% w/w.

120. The method according to any one of claims 79-119, wherein the second surfactant comprises from 2% w/w to about 10% w/w of the total weight of composition.

121. The method of claim 120, wherein the second surfactant comprises from 2% w/w to about 6% w/w.

122. The method of claim 121, wherein the second surfactant comprises from 3% w/w to about 5% w/w.

123. The method according to any one of claims 80-122, wherein the thermal process is hot melt extrusion and the screw speed of the of the extruder is from about 10 rpm to about 500 rpm.

124. The method of claim 123, wherein the screw speed is from about 50 rpm to about 250 rpm.

125. The method according to any one of claims 79-124, wherein the thermal process comprises heating the composition from a temperature of about 60° C. to about 220° C.

126. The method of claim 125, wherein the temperature is from about 100° C. to about 200° C.

127. The method according to any one of claims 79-126, wherein the thermal process comprises heating the composition for a time period from about 2 minutes to about 3 hours.

128. The method of claim 127, wherein the time period is about 5 minutes to about 1 hour.

129. The method according to any one of claims 79-128 further comprising milling the pharmaceutical composition.

130. The method according to any one of claims 79-129 further comprising sieving the pharmaceutical composition.

131. The method of claim 130, wherein the pharmaceutical composition is sieved through a screen with a pore size from about 250 μm to about 1000 μm.

132. The method according to any one of claims 79-131, wherein the method is substantially free of a solvent.

133. The method of claim 132, wherein the method is essentially free of a solvent.

134. The method according to any one of claims 79-133, wherein the therapeutic agent has a solubility in water of less than 5 mg/mL.

135. The method of claim 134, wherein the therapeutic agent is a Biopharmaceutics Classification System Class II or IV compound.

136. The method according to any one of claims 79-135, wherein the therapeutic agent is known to undergo thermal degradation.

137. The method according to any one of claims 79-136, wherein the therapeutic agent is not albendazole, benomyl, benzimidazole fungicide, carbendazim, ciclobendazole, fenbendazole, flubendazole, mebendazole, nocodazole, oxfendazole and oxibendazole.

138. The method according to any one of claims 79-137, wherein the pharmaceutically acceptable polymer is a cellulosic polymer.

139. The method of claim 138, wherein the pharmaceutically acceptable polymer is a neutral cellulosic polymer.

140. The method of claim 138, wherein the pharmaceutically acceptable polymer is an ionizable cellulosic polymer.

141. The method according to any one of claims 79-137, wherein the pharmaceutically acceptable polymer is a non-cellulosic polymer.

142. The method of claim 141, wherein the pharmaceutically acceptable polymer is a neutral non-cellulosic polymer.

143. The method of claim 141, wherein the pharmaceutically acceptable polymer is an ionizable non-cellulosic polymer.

144. The method of claim 141, wherein the pharmaceutically acceptable polymer is a polymethacrylate or polyacrylate functionalized with a carboxylic acid group.

145. The method according to any one of claims 79-144, wherein one or more of lipids, solvent, or oils is in the liquid phase.

146. The method according to any one of claims 79-145, wherein the spontaneously emulsifying composition comprises a lipid or oil.

147. The method of claim 146, wherein the lipid or oil is an ester of a fatty acid.

148. The method of claim 147, wherein the lipid or oil is a glyceride ester of one, two, or three fatty acids.

149. The method of either claim 147 or claim 147, wherein the fatty acids is medium chain fatty acids.

150. The method of claim 149, wherein the lipid or oil is Capmul®.

151. The method according to any one of claims 79-150, wherein the spontaneously emulsifying composition comprises a solvent.

152. The method of claim 151, wherein the solvent is a hydrophobic solvent.

153. The method of either claim 151 or claim 152, wherein the solvent contains one or more aromatic groups.

154. The method according to any one of claims 151-153, wherein the solvent is benzyl benzoate.

155. The method according to any one of claims 79-154, wherein the spontaneously emulsifying composition comprises a surfactant or hydrophilic solvent that contains one or more polyethylene glycol or polypropylene glycol repeating units.

156. The method of claim 155, wherein the hydrophilic solvent is a PEG polymer.

157. The method of claim 156, wherein the hydrophilic solvent is a PEG polymer with a molecular weight from 100 Daltons to 2000 Daltons.

158. The method of claim 157, wherein the hydrophilic solvent is PEG 200 or PEG 400.

159. The method according to any one of claims 79-158, wherein the pharmaceutical composition comprises a first surfactant.

160. The method of claim 159, wherein the first surfactant is polyethoxylated castor oil.

161. The method of claim 160, wherein the first surfactant is Cremophor EL.

162. The method according to any one of claims 79-161, wherein the pharmaceutical composition further comprises a second surfactant.

163. The method of claim 162, wherein the second surfactant is a compound with a hydrophobic component and a PEG or polypropylene glycol component.

164. The method of claim 163, wherein the hydrophobic component is a fatty acid.

165. The method of either claim 163 or claim 164, wherein the PEG or polypropylene glycol component is a PEGylated polysorbate.

166. The method according to any one of claims 162-165, wherein the second surfactant is Tween®.

167. The method according to any one of claims 79-166, wherein the composition further comprises an excipient.

168. The method of claim 167, wherein the excipient is a lubricant, disintegrant, binder, filler, surfactant, or any combination thereof

169. A pharmaceutical composition prepared according to methods of any one of claims 79-168.

170. The pharmaceutical composition according to any one of claims 1-78 and 169, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, in lipid compositions, via a catheter, via a lavage, via continuous infusion, via infusion, via inhalation, via injection, via local delivery, or via localized perfusion.

171. The pharmaceutical composition of claim 170, wherein the pharmaceutical composition is formulated for oral administration.

172. The pharmaceutical composition of either claim 170 or claim 171, wherein the pharmaceutical composition is formulated as a hard or soft capsule, a tablet, a syrup, a suspension, an emulsion, a solution, or a wafer.

173. A method of treating a disease or disorder in a patient in need thereof comprising administering to the patient a therapeutically effective amount of a pharmaceutical composition according to any one of claims 1-78 and 169-172 comprising a therapeutic agent effective to treat the disease or disorder.

174. A method of preventing a disease or disorder in a patient in need thereof comprising administering to the patient a therapeutically effective amount of a pharmaceutical composition according to any one of claims 1-78 and 169-172 comprising a therapeutic agent effective to prevent the disease or disorder.
